(19) United States
(12) Patent Application Publication
(10) Pub. No.: US 2025/0200599 A1
(43) Pub. Date: Jun. 19, 2025

(54) SYSTEMS AND METHODS FOR MANAGEMENT OF HEALTHCARE SERVICES

(71) Applicant: Mariah BOWLES, Florissant, MO (US)

(72) Inventor: Mariah BOWLES, Florissant, MO (US)

(21) Appl. No.: 18/846,207

(22) PCT Filed: Feb. 8, 2023
(86) PCT No.: PCT/US23/62169

§ 371 Date: Sep. 11, 2024

Related U.S. Application Data
(60) Provisional application No. 63/319,019, filed on Mar. 11, 2022.

(51) Int. Cl. G06Q 30/0201; G06Q 30/08; G06Q 40/08; G16H 10/60; G16H 40/20; G16H 80/00

(52) U.S. Cl. CPC G06Q 30/0206; G06Q 30/08; G06Q 40/08; G16H 10/60; G16H 40/20; G16H 80/00

(57) ABSTRACT

Systems and methods for management of healthcare services include a processor and a memory communicatively coupled to the processor. The memory stores instructions that, when executed by the processor, perform operations, including identifying healthcare providers from a list of healthcare providers that are authorized to perform healthcare services associated with at least one healthcare concern for a patient, receiving insurance information associated with the patient, receiving bids from the healthcare providers for the healthcare services, determining out-of-pocket expenses for the patient for the healthcare services based on the insurance information and the bids by the healthcare providers, receiving an approval or rejection from the patient of at least one bid submitted by the healthcare providers, and sending an appointment booking to the patient and the respective healthcare provider if the patient approves the bid submitted by the healthcare provider.

### CROSS-REFERENCE TO RELATED APPLICATION

This application claims priority to U.S. Provisional Application No. 63/319,019 filed Mar. 11, 2022, the content of which is incorporated herein by reference in its entirety.

### BACKGROUND

The field of the disclosure relates generally to systems and methods for healthcare services, and, more specifically, to a system and method that enables patients and healthcare providers to search, schedule, and pay for healthcare services.

Patients usually have one or more healthcare providers that they utilize for healthcare services. Typically, a patient or a representative of the patient contacts the healthcare provider and sets up an appointment when the patient is in need of healthcare services. The patient then travels to the healthcare provider's location at the appointment time and waits at the location for the healthcare provider to provide services. Sometimes, however, the healthcare provider is unavailable for the healthcare services required by the patient. In addition, the patient may not be able to locate alternative healthcare providers because of restrictions such as insurance requirements, cost, inability to travel to other locations, or ignorance of other healthcare providers. As a result, at least some patients may not receive the healthcare services that they need. Moreover, patients often do not receive information regarding costs of healthcare services prior to the appointments and may not be able to make informed decisions regarding the costs of their healthcare services. Also, the healthcare providers may not provide competitive pricing because the patients do not have alternative options for healthcare services. As a result, the consumer experience for healthcare services is not satisfactory and costs for healthcare services have increased.

### BRIEF DESCRIPTION

In one aspect, a method for managing a healthcare services account includes receiving login information from a user and retrieving from a memory at least one characteristic of the user based on the login information. The at least one characteristic is associated with a user account designation. The method also includes determining the user account designation based on the at least one characteristic of the user. The at least one characteristic comprises at least one of an age, a mental capacity, a guardianship status, a grade level, a dependent status, insurance coverage, and an employment status. The user account designation comprises one of a primary account and a secondary account. Each secondary account is associated with at least one primary account. The method further includes providing a list of healthcare services available for the user based on the user account designation, receiving a selection of at least one healthcare service from the user, and requesting approval from the primary account if the associated secondary account requests a healthcare service that requires approval from the primary account for the secondary account to select the healthcare service. The method also includes providing access to the selected healthcare service if the user is authorized for the healthcare service based on the user account designation or if the selection is approved by the associated primary account for the secondary account and receiving at least one input from the user related to the selected healthcare service. The input from a secondary account is monitored for indications of potentially hazardous situations. The method further includes notifying the associated primary account if the inputs from the secondary account indicate a potentially hazardous situation.

In another aspect, a method for allocating resources for healthcare services includes generating a map including a plurality of healthcare appointments. Each healthcare appointment includes a patient, a healthcare service, a timeframe, a location, and a status. The status includes assigned, unassigned, or completed. The method also includes identifying a healthcare service provider that is associated with the healthcare service included in at least one healthcare appointment, comparing a location of the healthcare service provider to the location of the at least one healthcare appointment, and determining an availability of the healthcare service provider during the time frame of the healthcare appointment. The method further includes assigning the healthcare appointment to the healthcare service provider if the healthcare service provider is available during the time frame of the healthcare appointment and within an area of the location of the at least one healthcare appointment, and updating the map to indicate that the status of the at least one healthcare appointment is assigned.

In yet another aspect, a method includes receiving a search request from a patient for at least one healthcare concern, retrieving a list of healthcare providers associated with the healthcare concern from a database of healthcare providers, providing to the patient a list of healthcare services provided by the healthcare providers and a comparison of costs associated with the healthcare services provided by the healthcare providers, and providing to the patient a list of available appointments for each healthcare provider. The method also includes receiving a selection from the patient including a selected healthcare provider, a selected healthcare service, and a selected appointment from the list of available appointments. The selected healthcare service is associated with a cost from the comparison of costs. The method further includes generating an appointment booking based on the selection. The appointment booking includes the selected appointment, the selected healthcare service, the selected healthcare provider, and the cost associated with the healthcare service. The method also includes sending the appointment booking to the healthcare provider, receiving confirmation from the patient that the appointment has occurred, and accepting payment from the patient of the cost associated with the selected healthcare service after receiving confirmation from the patient that the appointment has occurred.

In still another aspect, a system includes a processor and a memory communicatively coupled to the processor. The memory stores instructions that, when executed by the processor, perform operations, including receiving a search request from a patient for at least one healthcare concern, retrieving a list of healthcare providers associated with the healthcare concern from a database of healthcare providers, and providing to the patient a list of healthcare services provided by the healthcare providers and a comparison of costs associated with the healthcare services provided by the healthcare providers. The operations also include providing to the patient a list of available appointments for each healthcare provider, and receiving a selection from the patient including a selected healthcare provider, a selected healthcare service, and a selected appointment from the list of available appointments. The selected healthcare service is associated with a cost from the comparison of costs. The operations further include generating an appointment booking based on the selection. The appointment booking includes the selected appointment, the selected healthcare service, the selected healthcare provider, and the cost associated with the healthcare service. The operations additionally include sending the appointment booking to the healthcare provider, receiving confirmation from the patient that the appointment has occurred, and accepting payment from the patient of the cost associated with the selected healthcare service after receiving confirmation from the patient that the appointment has occurred.

In another aspect, a healthcare marketplace system includes a processor and a memory communicatively coupled to the processor. The memory stores instructions that, when executed by the processor, perform operations, including identifying healthcare providers from a list of healthcare providers that are authorized to perform healthcare services associated with at least one healthcare concern for a patient, receiving insurance information associated with the patient, receiving bids from the healthcare providers for the healthcare services, determining out-of-pocket expenses for the patient for the healthcare services based on the insurance information and the bids by the healthcare providers, receiving an approval or rejection from the patient of at least one bid submitted by the healthcare providers, and sending an appointment booking to the patient and the respective healthcare provider if the patient approves the bid submitted by the healthcare provider.

### BRIEF DESCRIPTION OF THE DRAWINGS

FIGS. 1A-1E illustrate an example system for managing healthcare services, the system including a healthcare management system in communicative connection with at least one healthcare provider device and a user device.

FIG. 2 illustrates an example database for use with the system shown in FIGS. 1A-1E.

FIG. 3 illustrates a flow diagram of an example method of operating a computer system for managing healthcare services, such as the system shown in FIG. 1.

FIG. 4 illustrates a flow diagram of an example method of operating a computer system for providing remote healthcare services, such as the system shown in FIG. 1.

FIG. 5 illustrates an example user account system for use with the system shown in FIGS. 1A-1E.

FIG. 6 illustrates a flow diagram of an example method of operating a user account system such as the system shown in FIG. 5.

FIG. 7 illustrates an example healthcare provider management system for use with the system shown in FIGS. 1A-1E.

FIG. 8 illustrates an example map for use with the healthcare provider management system shown in FIG. 7.

FIG. 9 illustrates a flow diagram of an example method of operating a healthcare provider management system such as the system shown in FIG. 7.

FIG. 10 illustrates an example computing device.

FIGS. 11A and 11B illustrate an example of another system for managing healthcare services.

FIGS. 12A-12C illustrate an example of yet another system for managing healthcare services.

### DETAILED DESCRIPTION

As used herein, an element or step recited in the singular and preceded with the word “a” or “an” should be understood as not excluding plural elements or steps, unless such exclusion is explicitly recited. Furthermore, references to “example” or “one implementation” of the present disclosure are not intended to be interpreted as excluding the existence of additional implementations that also incorporate the recited features.

As used herein, the term “non-transitory computer-readable media” is intended to be representative of any tangible computer-based device implemented in any method of technology for short-term and long-term storage of information, such as, computer-readable instructions, data structures, program modules and sub-modules, or other data in any device. Therefore, the methods described herein may be encoded as executable instructions embodied in a tangible, non-transitory, computer-readable medium, including, without limitation, a storage device and/or a memory device. Such instructions, when executed by a processor, cause the processor to perform at least a portion of the methods described herein. Moreover, as used herein, the term “non-transitory computer-readable media” includes all tangible, computer-readable media, including, without limitation, non-transitory computer storage devices, including without limitation, volatile and non-volatile media, and removable and non-removable media such as firmware, physical and virtual storage, CD-ROMS, DVDs, and any other digital source such as a network or the Internet, as well as yet to be developed digital means, with the sole exception being transitory, propagating signal.

The present disclosure provides systems and methods for managing healthcare services. Embodiments facilitate a user searching for healthcare services based on a healthcare concern. The user is able to compare costs and healthcare services from multiple healthcare providers to make an informed decision. The user is able to obtain healthcare services from healthcare providers that are at remote locations. For example, the healthcare providers may be experts in a field, leading researchers on the user's healthcare concern, and/or may be at a location that has a high incidence for the healthcare concern. As result, the patient is able to receive the best possible care in a cost-effective manner.

Systems and methods facilitate healthcare service providers allocating resources to efficiently and timely provide healthcare services such as at-home healthcare services for patients. For example, the systems may include a map with appointment information. The map facilitates assigning available healthcare providers to appointments within a local area. Moreover, the system and methods provide access in a protected manner to healthcare services for minors or patients with reduced or limited capacity. For example, the systems and methods provide a platform with primary and secondary user accounts that facilitate users, such as minors or other patients, researching, scheduling, and receiving healthcare services that would otherwise be difficult for the patients to access.

As a result, the system and methods facilitate patients receiving timely healthcare services in a manner that is beneficial for the users and the healthcare providers. Moreover, the costs of the healthcare services may be decreased because 1) the users can compare costs before selecting services, 2) the healthcare providers are incentivized to provide competitive pricing, and 3) the healthcare providers can better allocate their resources and reduce expenses.

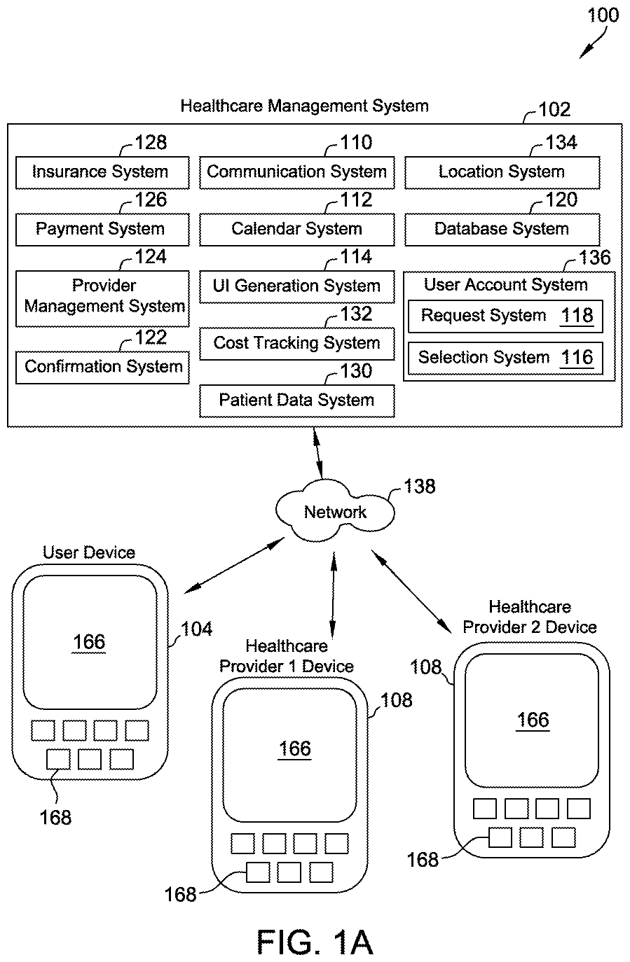

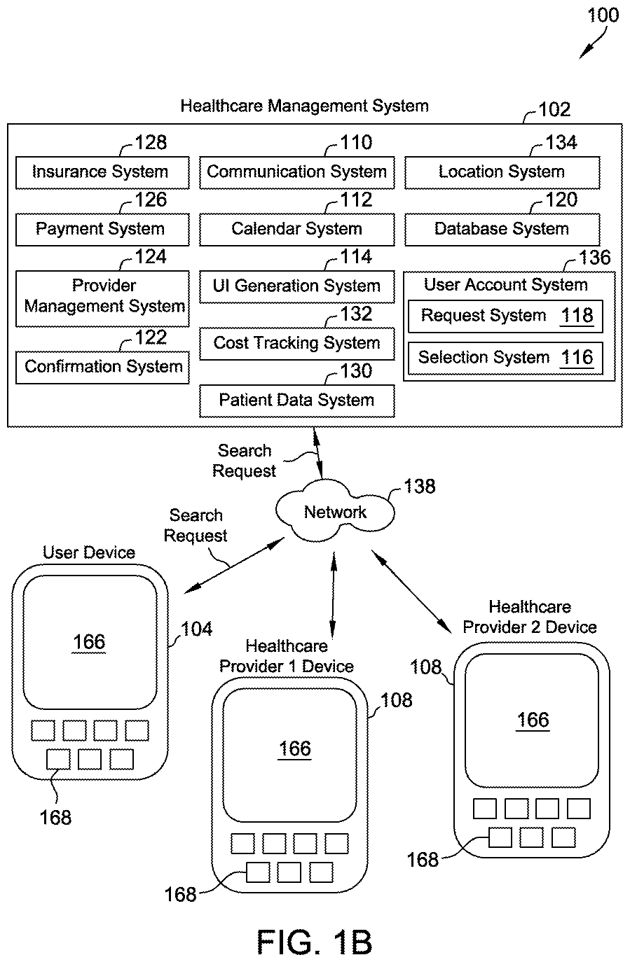

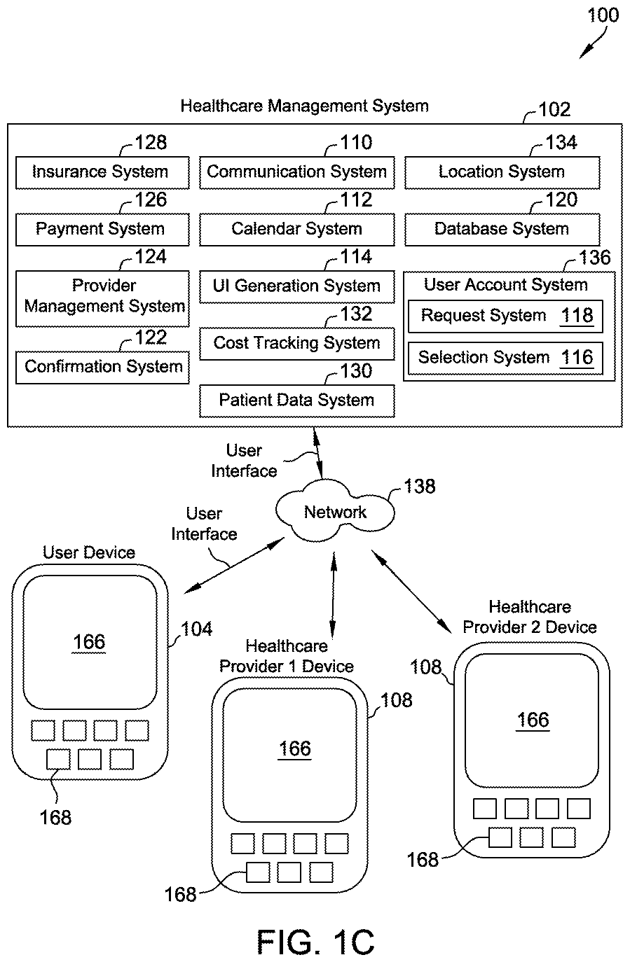

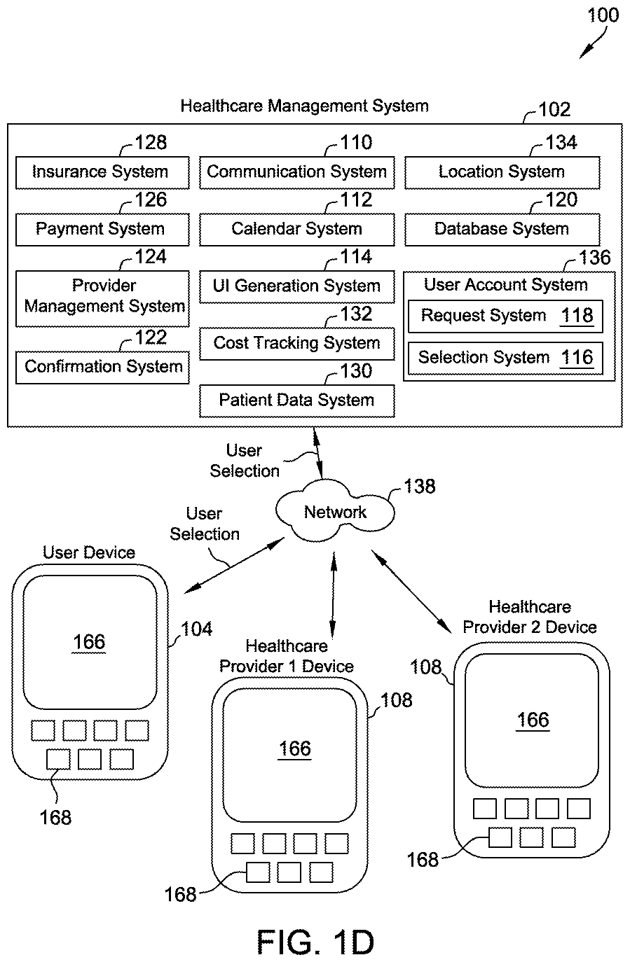

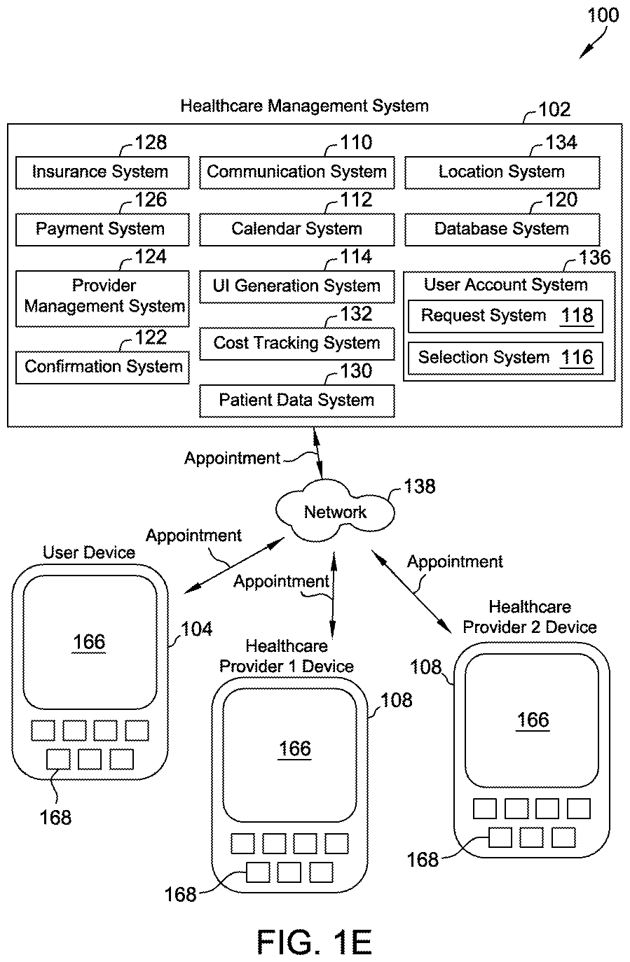

FIGS. 1A-IE illustrate an example system 100 for managing healthcare services. The system 100 includes a healthcare management system 102, a user device 104, and at least one healthcare provider device 108. The healthcare management system 102 includes a communication system 110, a calendar system 112, a user-interface (UI) generation system 114, a selection system 116, a request system 118, at least one database 120, a confirmation system 122, a provider management system 124, a payment system 126, an insurance system 128, a patient data system 130, a cost tracking system 132, a location system 134, and an account system 136. The healthcare management system 102 may be communicatively coupled to the user device 104 and/or the healthcare provider device 108 via a network 138. Although a network 138 is specifically shown and described, the healthcare management system 102 may be communicatively coupled to the user device and/or to the healthcare provider devices 108 through various communication protocols including, but not limited to, Bluetooth, near-field communication, or other wireless (or wired) communication protocols.

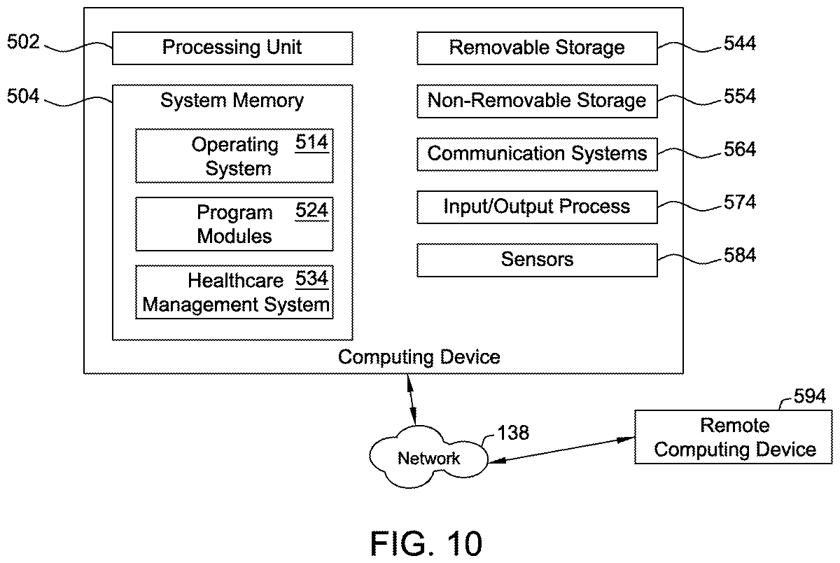

Additionally, although the healthcare management system 102, the user device 104, and the healthcare provider devices 108 are shown as being separate from each other, one or more of the healthcare management system 102, the user device 104, and/or the healthcare provider devices 108 may be part of the same device or otherwise integrated together. For example, any of the healthcare management system 102, the user device 104, and/or the healthcare provider devices 108 may be incorporated into a computing device 500 (shown in FIG. 10). The system 100 may include any number of computing devices 500.

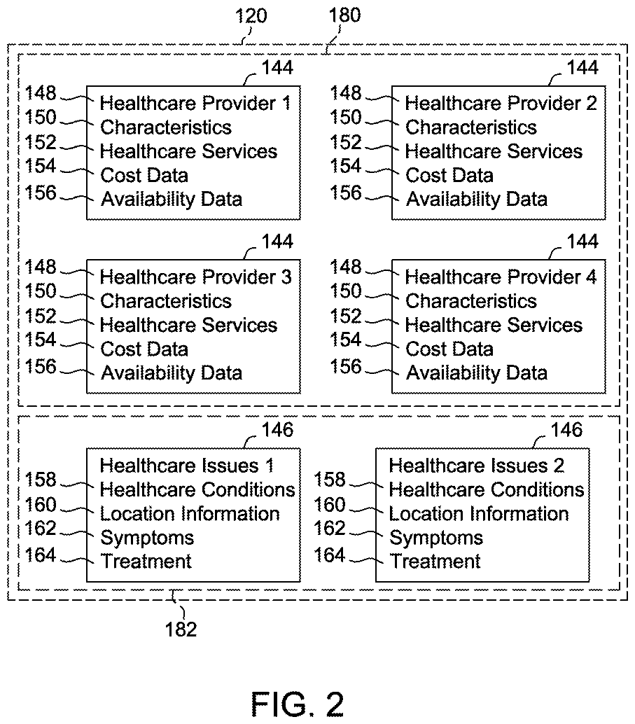

FIG. 2 illustrates an example database 120 for use with the system 100. The database 120 includes a plurality of healthcare provider files 144 and healthcare information files 146. For example, each healthcare provider file 144 includes a healthcare provider 148, a characteristic 150 associated with the healthcare provider, healthcare services 152 provided by the healthcare provider, cost data 154 associated with the healthcare services 152, and availability data 156. For example, the characteristic 150 may include a name, a full-time or part-time designation, an experience level, a performance rating, a limit on a number of appointments, a priority ranking, and/or license or certification information for the healthcare provider. Each healthcare information file 146 includes, for example, healthcare conditions 158, location information 160, symptoms 162, and treatments 164.

The healthcare provider files 144 and the healthcare information files 146 may be provided and/or maintained by a business, a hiring service, the healthcare providers, government agencies, and/or other authoritative entities. The healthcare providers 148 may include employees of a business, independent contractors, employees of a hiring service, freelance workers, consultants, and/or any other workers. Confidential information of the healthcare providers 148 is protected because the information is maintained in the database 120 and access to information in the database 120 may be restricted from the administrators and/or other healthcare providers. For example, the healthcare management system 102 retrieves the information from the database 120 such as characteristics associated with the healthcare providers 148 and can automatically make determinations such as if the healthcare provider is qualified to provide desired healthcare services without input from an administrator.

Referring to FIG. 1A, the user device and the healthcare provider device 108 each include a display device 166 and an input device 168. In some embodiments, the input device 168 includes a keyboard, a computer pointer device, a touch screen, a microphone, a camera, and/or any other suitable input device. The input device 168 may be incorporated into a computing device (e.g., the computing device 500) that includes a processor and/or the display device 166. In other embodiments, the input device 168 may be located remotely from the computing device 500 and/or incorporated into a computing device that communicates with the computing device 500 using a computer network. For example, in some embodiments, the input device 168 is included on a mobile computing device (e.g., a smartphone or a tablet computing device).

In some examples, the user device 104 and/or the healthcare provider device 108 require user authentication. For example, the devices 104, 108 may require login information including but not limited to a personal identifier, an authentication code, a password, a fingerprint, a verbal cue, an answer to a security question, two factor authentication, a security token, and/or any other suitable authentication means. The user device 104 and the healthcare provider device 108 may provide the login information to the user account system 136 and/or the provider management system 124 for logging into respective accounts.

With reference to FIG. 1B, the request system 118 is configured receive a search request regarding healthcare services from a user entered via the input device 168 of the user device 104. The request system 118 retrieves healthcare services and/or healthcare providers based on the search request. For example, the retrieved healthcare services and/or healthcare providers are filtered based on real-time updates of the characteristics of the healthcare services and/or healthcare providers such as their availability. In some examples, the UI generation system 114 provides a user interface for the user device, as shown in FIG. 1C. For example, the user may submit a request for a healthcare appointment within a designated timeframe. The request system 118 retrieves a list of healthcare providers from the database 120 that are available to provide the healthcare service. The UI generation system 114 provides a user interface to display the list of healthcare providers.

As illustrated in FIG. 1D, the selection system 116 receives a selection of at least one healthcare service or at least one healthcare service provider that is inputted by the user via the input device 168 of the user device 104. The selection system 116 then generates, in real-time, an appointment based on the selection and provides the appointment to the calendar system 112 and the communication system 110. The calendar system 112 may track the availability of the healthcare providers and the appointment timeframes.

With reference to FIG. 1E, the communication system 110, in real-time, sends information relating to the appointment to the healthcare service provider device 108 and the user device 104. The patient data system 130 records patient information relating to the healthcare service in the memory and provides access to the information to authorized users prior to, during, or after the healthcare service appointment. For example, the patient data system 130 may provide patient records to the healthcare service providers for a time frame encompassing the healthcare appointment.

The location system 134 may monitor and record locations of the healthcare service providers and/or the users. For example, the location system 134 may receive location information using location services on the user device 104 and/or the healthcare provider device 108. The location system 134 may restrict activity of the healthcare service providers and/or the users to when the healthcare service providers and/or the users are within specified geographic boundaries. For example, the location system 134 may enable a user to confirm, via the confirmation system 122, that the healthcare service appointment is starting or completed only when the healthcare service provider is at a specified location. The healthcare service provider may request, via the confirmation system 122, that the user confirm that the appointment is starting or completed when the healthcare service provider is at the location.

The confirmation system 122 may record an attendance or non-attendance of the patient for scheduled appointments in the patient data system 130 and the selection system 116 may restrict the patient's access to select appointments if the patient has one or more scheduled appointments that the patient did not attend. For example, the user may be prohibited from selecting remote appointments if the patient has a history of non-attendance. In some examples, the users will be charged cancellation fees for cancelling or changing appointments after scheduling.

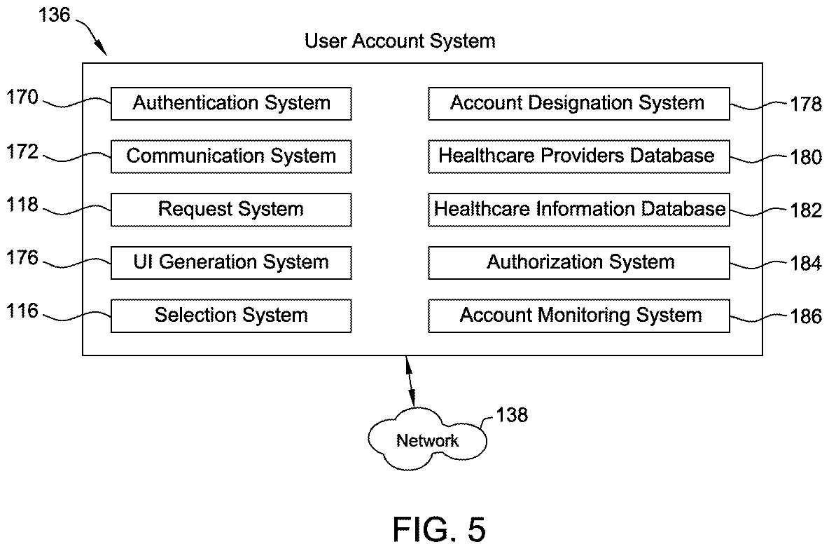

The user account system 136 facilitates users accessing the healthcare management system with different levels of authorized use. For example, the user account system 136 provides primary and secondary user accounts. The secondary user accounts are associated with primary user accounts and require authorization for at least some healthcare services. An example user account system 136 is described in relation to FIG. 5.

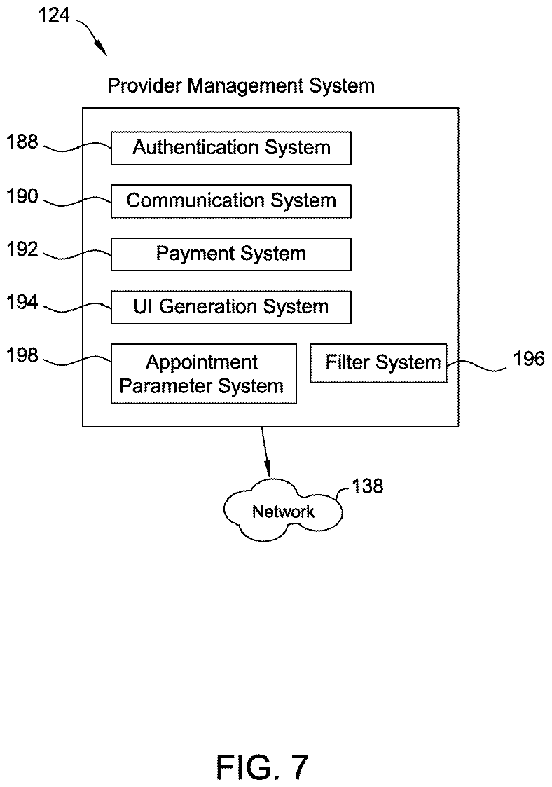

The provider management system 124 manages the healthcare service providers, facilitates the healthcare service providers providing appropriate healthcare services, and processes payment transfers for the healthcare service providers. For example, the provider management system 124 verifies that the healthcare service providers are licensed to provide healthcare services. An example provider management system 124 is described in relation to FIG. 7.

The cost tracking system 132 determines costs for healthcare services selected via the selection system 116. The cost tracking system 132 determines any copays or deposits that are required before the healthcare service appointment. In addition, the cost tracking system 132 determines amounts that are owed by the patient, an insurance provider, and/or a responsible agency. The payment system 126 receives payment from the user, an insurance provider, and/or other responsible agency for any deposit before the appointment. The cost tracking system 132 determines final costs when the healthcare services are confirmed via the confirmation system 122. The payment system 126 collects payment after the user confirms that is complete via the confirmation system. For example, the cost tracking system 132 determines a cost for the healthcare services that are actually provided during the healthcare service appointment after receiving confirmation, in real-time, of the completed appointment. The communication system 110 sends an invoice for the costs to the appropriate party and the payment system 126 receives payments.

The insurance system 128 may receive and record insurance information for a patient. The insurance information may include a policyholder name, insurance provider information, co-pays, coverages, exclusions, and network information. The insurance system 128 provides the insurance information to the cost tracking system 132 for determining costs. In addition, the insurance system 128 provides the insurance information to the communication system 110 for contacting the insurance provider for invoices, authorizations for healthcare services, and requests for information. As a result, the insurance system 128 provides a centralized database of a patient's insurance information that can be provided to multiple healthcare service providers for different healthcare services. Accordingly, a user is only required to update insurance information at a single location by inputting the insurance information to the insurance system 128 via the user device 104 and the user does not need to repeatedly provide insurance information for each healthcare service provider and healthcare service. Moreover, in some embodiments, the insurance system 128 automatically retrieves insurance information from an insurance provider database when the user's identity is authenticated.

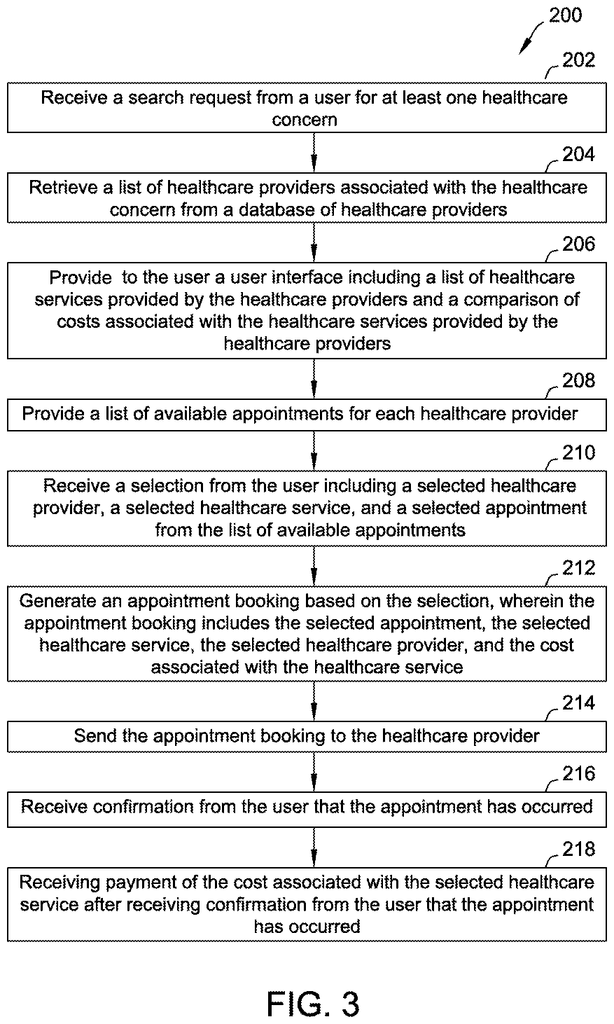

FIG. 3 is a flow diagram of an example method 200 of operating a system for managing healthcare services, such as the system 100 shown in FIG. 1A. Referring to FIGS. 1A-3, the method 200 includes receiving 202 a search request from a computing device associated with a user for at least one healthcare concern and retrieving 204 a list of healthcare providers associated with the healthcare concern from the database 120 of healthcare providers. For example, the request system 118 receives the search request input by the user via the input device 168 of the user device 104. In some embodiments, the user may be a patient and/or someone authorized to search on behalf of the patient. For example, the user may be a medical proxy for a patient and/or an agency such as an insurance company. The patient or someone acting on the patient's behalf may request the agency to run the search because the agency as information such as insurance and/or service agreements that facilitate searching for an appropriate provider. The agency may run results and filter results for the user and/or select one or more providers for the user.

The search request may include symptoms, a requested healthcare service, a medication, a healthcare provider, and/or any other information related to a healthcare concern. The request system 118 determines, in real-time, healthcare services and/or healthcare providers that are relevant based on the search request. For example, the request system 118 identifies key terms or phrases in the search request and compares the key terms to a database of terms that are related to healthcare services. The request system 118 identifies the healthcare service that is most closely related to the search request and retrieves the list of healthcare providers that are associated with the healthcare service. In some embodiments, the user selects the healthcare services from a menu and the request system 118 retrieves the healthcare providers for the selected healthcare service.

In some embodiments, the request system 118 receives from a computing device associated with the user a request for bids for a healthcare service. After receiving the bid request, the request system 118 may determine a healthcare service selected by the user and then determine healthcare providers that are associated with the healthcare service. The request for bids may also include insurance information and payment information. The request system 118 sends the request to the healthcare providers and/or retrieves prerecorded bidding information from the healthcare providers. The request system 118 receives at least one bid from the healthcare providers for the healthcare service. For example, the bid from the healthcare provider may include at least an appointment availability and a cost. The user may select or reject bids via the selection system 116.

The UI generation system 114 provides 206 to a computing device associated with the user a user interface including a list of healthcare services provided by the healthcare providers and a comparison of costs or bids associated with the healthcare services provided by the healthcare providers. The list of healthcare services may be sorted and/or filtered based on the costs. Accordingly, the user is presented with costs prior to selecting a healthcare service or a healthcare provider and the user is able to make an informed decision regarding the healthcare service. Moreover, the healthcare providers are incentivized to provide competitive pricing because the user is able to compare costs before selecting services. In addition, the list of healthcare services and/or providers may be sorted or filtered based on insurance information or other parameters. Therefore, the user may identify healthcare providers that accept or work with specified insurance providers. In some embodiments, the provider management system 124 determines a service history and license status of each healthcare provider in the database and the list of healthcare providers includes only healthcare providers with an active license status and a positive service history.

The calendar system 112 provides 208 a list of available appointments for each healthcare provider that is provided to or selected by the user. For example, the calendar system 112 provides a list of appointments to the UI generation system 114 and the UI generation system 114 generates a user interface including a calendar or chart of the appointments. The selection system 116 receives 210 a selection from the user including a selected healthcare provider, a selected healthcare service, and a selected appointment from the list of available appointments. The cost tracking system 132 associates the selected healthcare service with a cost from the comparison of costs and records the associated cost for the selected appointment. In some embodiments, the cost tracking system 132 determines a copay or deposit that is required from the user before the appointment. The copay may be determined based on insurance information from the insurance system 128. A deposit may be determined based on an attendance history. For example, the user may be required to pay a deposit if the user has a record of missed appointments. Alternatively, a deposit may be waived if the user has a history of minimal missed appointments or no missed appointments.

In some embodiments, the cost tracking system 132 provides, via the communication system 110, the costs associated with the healthcare services to a third party (such as an insurance provider or a primary account) and obtains authorization from the third party for the costs before providing to the patient the list of healthcare services provided by the healthcare providers. Accordingly, the user can select the healthcare services without having to check for or consider third party authorization.

The calendar system 112 generates 212 an appointment booking based on the selection. The appointment booking includes the selected appointment, the selected healthcare service, the selected healthcare provider, and the cost associated with the healthcare service. The communication system 110 sends 214 the appointment booking to the healthcare provider. In some embodiments, the calendar system 112 updates in real-time a list of available appointments for a healthcare provider based on the selected appointment.

In some embodiments, the communication system 110 provides a communication link between the user or patient and the healthcare service provider for the appointment. For example, the communication system 110 facilitates the user or patient exchanging information with the healthcare service provider. In some embodiments, the communication system 110 provides a virtual meeting platform for the healthcare service provider to provide the healthcare services at a location remote from the patient. The communication system 110 may send a notification to the user or patient that the healthcare provider has arrived at a location for the healthcare services. In addition, the communication system 110 may receive verification from the patient that the healthcare provider has arrived at the location.

After or during the appointment, the confirmation system 122 receives 216 confirmation from the user that the appointment has occurred. For example, in some embodiments, the confirmation system 122 receives a patient-specific identification code from the patient and an input from the patient that confirms that the selected healthcare services were provided. In some embodiments, the confirmation system 122 receives a notification from the healthcare provider that the healthcare services have been completed. The confirmation system 122 provides a verification option to the patient (e.g., requests the patient-specific authorization code) and receives confirmation from the patient that the healthcare services have been completed. Also, the confirmation system 122 may record an attendance or non-attendance of the patient for scheduled appointments in the patient data system 130. For example, the confirmation system 122 may receive notification from the healthcare service provider and/or automatically record non-attendance if the appointment confirmation is not received from the patient or healthcare service provider. The patient data system 130 may provide attendance information to the selection system 116. The selection system 116 may restrict the patient's access to and/or capability to select appointments if the patient has one or more scheduled appointments that the patient did not attend.

The payment system 126 receives 218 payment of the cost associated with the selected healthcare service after receiving confirmation from the user that the appointment has occurred. Accordingly, the user does not have to pay for the healthcare services before the healthcare services are provided. Moreover, the user may have reduced out-of-pocket costs because the healthcare providers are incentivized to provide competitive pricing and services and may waive deposits or other patient costs to the extent allowed by insurance policies. In addition, the system facilitates a payment system based on customer satisfaction and the final costs can be at least partially determined based on the user's satisfaction with the healthcare services. For example, the cost tracking system 132 may adjust costs associated with the healthcare services based on results of a consumer survey completed by the user after the healthcare services. The customer survey may be sent at the same time or after a confirmation request is sent to the user.

In some embodiments, the communication system 110 sends an order for a healthcare product from the healthcare provider to a healthcare product vendor after the appointment. For example, the communication system 110 may send a prescription for medication from the healthcare service provider to a pharmacy. The payment system 126 can accept payment for a cost of the healthcare product.

As a result, the described systems and methods provide a centralized platform in which the user is able to manage different healthcare services from multiple healthcare providers and receive competitive pricing. In addition, the user may be able to identify alternative healthcare providers and receive timely healthcare services if a previously used healthcare provider is unavailable. As a result, the user is able to manage healthcare services more easily and the costs of the healthcare services may be reduced.

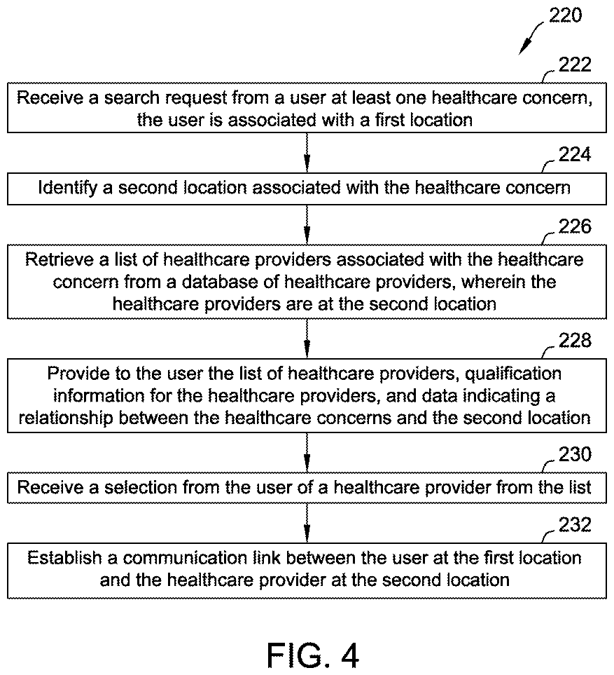

FIG. 4 is a flow diagram of an example method 220 of operating a system for managing remote healthcare services, such as the system 100 shown in FIG. 1. Referring to FIGS. 1A-2 and 4, the method 220 includes receiving 222 a search request from a computing device associated with a user for at least one healthcare concern. The request system 118 receives the search request. The location system 134 determines that the user is associated with a first location. For example, the location system 134 receives location information from the user device 104 associated with the user and/or location information inputted by the user and then relates a first location to the user based on the received information. In further embodiments, the location system 134 determines the first location based on information stored in a user profile associated with the user.

The location system 134 identifies 224 a second location associated with the healthcare concern. For example, the location system 134 identifies a second location that is associated with the healthcare concern based on information provided by the request system 118. The information provided by the request system 118 may be a healthcare service, a health condition, and/or a healthcare provider associated with the search request. The location system 134 identifies a relationship between the second location and the healthcare concern because, for example and without limitation, a number of incidents related to the healthcare concern are at the second location, research is generated at the second location and relates to the healthcare concern, and/or an expert in a field of the healthcare concern is at the second location.

The request system 118 retrieves 226 a list of healthcare providers associated with the healthcare concern from the database 120. For example, the request system 118 receives the second location from the location system 134 and retrieves the healthcare providers from the database that are at the second location and that provide healthcare services related to the healthcare concern.

The healthcare management system 102 provides 228 to a computing device associated with the user the list of healthcare providers, qualification information for the healthcare providers, and data indicating a relationship between the healthcare concern and the second location. For example, the relationship data may comprise a number of incidents related to the healthcare concern that are at the second location, research that is generated at the second location and relates to the healthcare concern, and/or an expert in a field of the healthcare concern that is at the second location. Qualification information may include the healthcare provider's licensure information and certification requirements for healthcare services at the second location. The qualification information may help the user appreciate if the healthcare provider at the second location will provide an appropriate standard of care even if the user is not at the second location or familiar with the second location. The UI generation system 114 generates a user interface including the information to be provided to the user.

The selection system 116 receives 230 a selection from a computing device associated with the user of a healthcare provider from the list. For example, the calendar system 112 may provide a list of available appointments for the healthcare provider and the user may select an available appointment using the selection system 116.

The communication system 110 establishes 232 a communication link between the user at the first location and the healthcare provider at the second location. For example, the communication system 110 may establish a channel for graphical or textual messages, an audio connection, and/or a video platform including a user interface and a provider interface for real-time communication between the user and the healthcare provider. The user at the first location may communicate with and receive healthcare services from the healthcare provider at the second location. In some embodiments, the provider management system 124 provides identity verification information to the user for the user to verify the identity of the selected healthcare provider when the communication link is established. The identify verification information may include at least one of a photo and credentials associated with the healthcare provider. For example, the system may retrieve verification information from employee records, government databases (e.g., DMV records), and/or any other suitable database. In some embodiments, the healthcare provider checkouts assigned equipment (e.g., a vehicle and/or healthcare apparatus) and the assigned equipment is associated with verification information for the employee such that the verification information is sent to a central database and/or the patient when the assigned equipment and/or provider is at a location.

FIG. 5 is a block diagram illustrating components of the user account system 136. The user account system 136 includes an authentication system 170, a communication system 172, the request system 118, a UI generation system 176, the selection system 116, an account designation system 178, a healthcare provider database 180, a healthcare information database 182, an authorization system 184, and a monitoring system 186. In some embodiments, the healthcare provider database 180 and/or the healthcare information database 182 are included or incorporated with the database 120 shown in FIG. 1A. In some embodiments, the healthcare provider database 180 and/or the healthcare information database 182 are at least partly stored remotely. For example, the healthcare information database 182 may include information from databases of research institutes, government agencies, and/or healthcare providers.

The authentication system 170 receives information from the users to authenticate the identity of the users and authorizes the users to access the user account. For example, the authentication information may include a login and password. In some embodiments, the authentication process for the authentication system 170 is based on the authentication process for the user device 104. For example, the user may enter authentication information to access the user device 104 and the user device 104 may automatically enter authentication information that is stored on the user device into the authentication system 170. In other embodiments, the user inputs the login information for the authentication system 170 separately from the login information for the user device 104. The authentication system 170 may require two-factor authentication, a security code, or any other authentication protocol.

The account designation system 178 determines a status of the account (e.g., active or inactive) and a designation for the account. Example account designations includes primary, secondary, adult, child, restricted, viewing only, etc. In the example, the user accounts are designated as primary and secondary accounts. Each secondary account is associated with at least one primary account. The account designation system 178 determines the account designation when the authentication information for the authentication system 170 is received and, in some embodiments, limits the content that is provided by the UI generation system 176 based on the account designation. For example, the UI generation system 176 may generate an unrestricted user interface when the account designation system 178 indicates a primary account and generate restricted user interfaces when the account designation system indicates a secondary account. In a restricted user interface, some menus, options, or selections may be limited or restricted. For example, the UI generation system 176 may provide a menu of healthcare services and appointments that are available for the user based on the account designation. The unrestricted user interfaces may have more selections available for the user than the restricted user interfaces. In some embodiments, the secondary account may be able to view and request access for options that require authorization from the primary account. The authorization system 184 will send an authorization request for the secondary account to be granted access. In other embodiments, the secondary account can only select or view menus of options that have been preauthorized by the primary account.

The request system 118 receives a user request regarding at least one healthcare service. For example, the user may input via the user device 104 a request for information or a request for a healthcare service. The UI generation system 176 provides a user interface that displays search results. The selection system 116 receives a user's selection of the healthcare services and/or appointments provided by the healthcare provider. The communication system 172 sends and receives communications with the other devices such as the communication systems 110 of the system 100, the communication system 190 of the provider management system 124, the user device 104, and/or the healthcare provider devices 108.

The authorization system 184 determines if authorization is required for a selected healthcare service and requests authorization if necessary. For example, the authorization system 184 receives the account designation from the account designation system 178 and if the account designation is a secondary account, the authorization system 184 determines if the selected healthcare service requires authorization. The authorization system 184 sends, via the communication system 172, a request to the primary account to authorize the healthcare service for the associated secondary account if the healthcare service requires authorization. Some healthcare services for the secondary account may not require authorization from the primary account and may be kept confidential from the primary account. For example, children of ages 14-17 years old may have options to select and maintain some services confidential from the primary account for confidential and autonomous care for some healthcare services. The account designation system 178 may determine a special account designation for individuals in protected classifications, e.g., children in selected age groups, and the user account system maintains the protected information confidentially and separate from information that is accessible to the primary account.

In some embodiments, the authorization system 184 requests authorization for healthcare services based on the cost of the healthcare services for a secondary account. For example, the authorization system 184 may request authorization for healthcare services that are out-of-network for an insurance plan or that have associated costs above a threshold amount. In addition, in some embodiments, the authorization system 184 receives pre-authorization from a primary account for at least some healthcare services before the secondary account makes selections. For example, the primary account may select a certain number of services or a price range for healthcare services that the secondary account is authorized to select. In some embodiments, the primary account receives information regarding costs to be authorized for a healthcare service but does not receive other confidential information regarding the secondary account's healthcare services.

The monitoring system 186 monitors user inputs and activity based on the account designation provided by the account designation system 178. For example, the monitoring system 186 monitors inputs provided by users on primary and secondary accounts and determines if the inputs indicate a potentially hazardous situation for the user. For example, the user may input information related to self-harm, abuse, suicidal thoughts, aggressive actions towards others, or other potential hazardous situations. The monitoring system 186 may provide an alert to an associated primary account, a healthcare service provider, and/or a responsible agency regarding the potentially hazardous situation. In some embodiments, the monitoring system 186 may direct the user to helpful information or healthcare services if the monitoring system detects a potentially harmful situation.

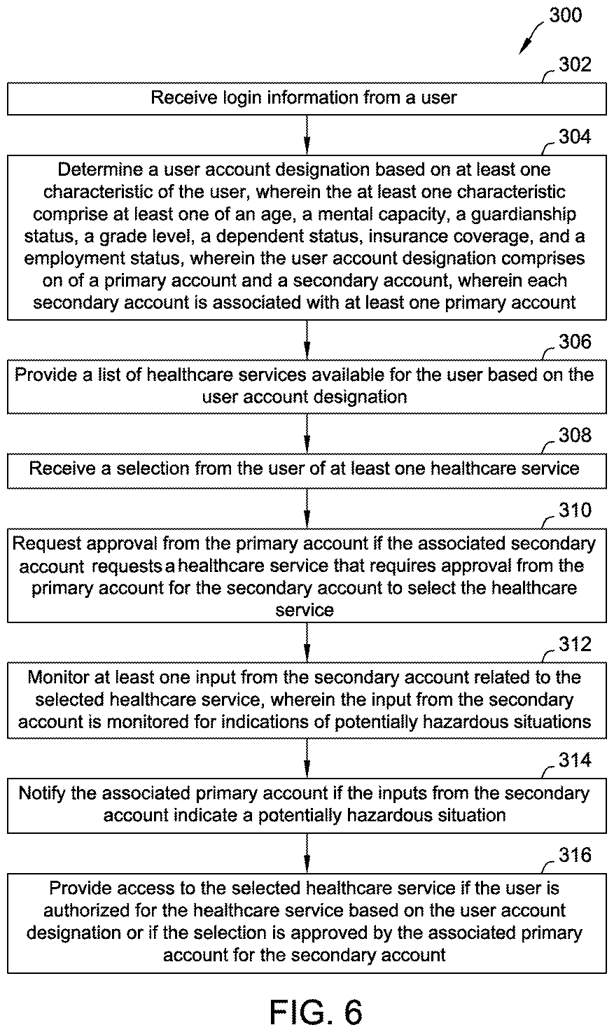

FIG. 6 is a flow diagram of an example method 300 of operating a system for managing healthcare services including user accounts. For example, the method 300 can be implemented using the system 100 shown in FIG. 1 and the user account system 136 shown in FIG. 5. Referring to FIGS. 1A, 5, and 6, the method 300 includes receiving 302 login information from a user. The login information may be inputted via the input device 168 of the user device 104 and received by the authentication system 170. The authentication system 170 confirms that the login information matches the login information for an account. For example, the login information may include a personal identifier and a password and/or an authentication code.

The account designation system 178 determines 304 a user account designation based on at least one characteristic of the user. For example, the account designation system 178 receives the login information from the authentication system 170 and retrieves at least one characteristic of the user from the database 120 based on the login information. For example, the characteristic may comprise at least one of an age, a mental capacity, a guardianship status, a grade level, a dependent status, insurance coverage, and an employment status. The account designation determines that the retrieved characteristic is associated with an account designation based on predetermined criteria. For example, a first account designation may be associated with users having ages under a threshold age (e.g., 18 years old) and a second account designation may be associated with users having ages at or above the threshold age. In some embodiments, the user account designation system 178 identifies the user account designation when the user account is first accessed or set up and the user account designation system records an indicator of the user account designation for retrieval upon subsequent logins. Accordingly, for subsequent access attempts, the user account designation system 178 determines 304 the user account designation by retrieving the stored indicator. In the example, the user account designation comprises one of a primary account and a secondary account. Each secondary account is associated with at least one primary account. For example, a primary account may be provided for a parent or legal guardian and a secondary account may be provided for a child or person with a legal guardian. The secondary account is associated with the primary account of the parent or legal guardian.

The user account system 136 provides 306 a list of healthcare services available for the user based on the user account designation. For example, the UI generation system 176 provides a user interface including the list of healthcare services that is displayed on the user device 104. The list of healthcare services may be sorted or filtered based on cost, healthcare providers, location, or any other suitable parameter. The list of healthcare services may be provided in a menu of the user interface. In some embodiments, the request system 118 receives a request from the user and generates the list of healthcare services in response to the request. In further embodiments, the request system 118 receives patient data associated with the user and automatically provides a healthcare suggestion to the user based on the patient data. For example, the request system 118 may retrieve the patient data when the authentication system 170 authenticates the login information and/or when the user account designation system 178 determines the account designation. In some embodiments, the patient data includes a patient location, and the healthcare suggestion is based on a healthcare issue that is associated with the patient location. The location information may be determined based on a global positioning system location, an IP address, or other location services information. In further embodiments, the patient data includes a medical condition, and the healthcare suggestion includes information or healthcare services relating to treatment of the medical condition. In some embodiments, the list of healthcare services is sorted based on the patient data. For example, the request system 118 may prominently position the healthcare services that are determined to be most relevant for the user based on the patient data. In some embodiments, for a secondary account, the list of healthcare services is sorted into healthcare services that require authorization and healthcare services that do not require authorization.

The selection system 116 receives 308 a selection from a computing device associated with the user of at least one healthcare service. For example, the selection system 116 receives an input provided by the user using the input device 168 of the user device 104. The authorization system 184 requests 310 approval from the primary account if an associated secondary account selects a healthcare service that requires approval from the primary account. For example, the authorization system 184 may request permission from a legal guardian (e.g., the primary account) for a minor (e.g., the secondary account) to receive specified healthcare services. In some embodiments, the authorization system 184 compares the healthcare selection to insurance information associated with the primary account or the secondary account, and requests approval from the primary account if the secondary account selects a healthcare service from a healthcare provider that is listed out-of-network based on the insurance information. In some embodiments, the authorization system 184 receives preapproval from the primary account for the secondary account to select at least one healthcare service that would otherwise require authorization.

The monitoring system 186 monitors 312 at least one input from the secondary account related to the selected healthcare service. For example, the input may include a search request for healthcare services, healthcare providers, and/or information related to healthcare services, a selection of at least one healthcare service, and/or a communication relating to the healthcare service. The monitoring system 186 monitors the inputs for indications of potentially hazardous situations. The monitoring system 186 notifies 314 the associated primary account if the inputs from the secondary account indicate a potentially hazardous situation or notifies a healthcare service provider, and/or a responsible agency if the inputs from a primary or secondary account indicate a potentially hazardous situation.

The authorization system 184 provides 316 access to the selected healthcare service if the user is authorized for the healthcare service based on the user account designation or if the selection is approved by the associated primary account for the secondary account. For example, the authorization system 184 allows the user to schedule an appointment for the healthcare service from a healthcare provider included in the healthcare provider database if the action is authorized. The authorization system 184 prevents a secondary account from scheduling a healthcare service that requires authorization without first receiving authorization from the primary account. In some embodiments, the request system 118 retrieves information related to the healthcare service from the healthcare information database 182 and provides the information to the user after the authorization system 184 indicates that the selected healthcare service is authorized. The appointment information and/or the retrieved information may be presented to the user in a user interface generated by the UI generation system 176.

The confirmation system 122 receives confirmation from a computing device associated with a patient that the healthcare service has been completed. The payment system 126 accepts payment from a computing device associated with the user of the cost associated with the selected healthcare service after receiving confirmation from the patient that the appointment has occurred. In some embodiments, the payment system 126 accepts payment from the user when the user is logged into the user account via the user account system. For example, the payment system 126 accepts payment from the primary account for healthcare services provided for the primary account or for a secondary account associated with the primary account. The payment system 126 receives payment from the secondary account if the selected healthcare service is included in a list of healthcare services that can be paid for by the secondary account. In addition, the payment system 126 may receive payment from a computing device associated with an insurance company of the insurance's companies share of the medical expenses.

FIG. 7 is a block diagram illustrating components of the provider management system 124. The provider management system 124 includes an authentication system 188, a communication system 190, a payment system 192, a UI generation system 194, a filter system 196, and an appointment parameter system 198. The provider management system 124 is configured to, for example, manage and monitor performance of the healthcare services by the healthcare providers, and handle payments to the healthcare providers.

The authentication system 188 receives information from the healthcare providers and verifies the accuracy and authenticity of the information. For example, the authentication system 188 receives inputs from each healthcare provider including business names, employees, credentials, licenses, financial data, tax information, and any other suitable data. The authentication system 188 compares the inputs to databases where available to verify accuracy. For example, the authentication system 188 may compare license information received from the healthcare provider to license information provided by regulatory authorities to confirm that the inputted information is accurate and the license is active. The authentication system 188 determines if all required information has been provided and requests additional information from the healthcare provider as necessary. In some embodiments, the authentication system 188 continuously monitors and updates the authentication information to ensure the authentication information is current. For example, the authentication system 188 may regularly retrieve license information from the regulatory authorities to ensure the license is active and that no new incidents have been reported. The authentication system 188 provides authorization for the healthcare providers to be included in the system and provide one or more healthcare services if the authentication system 188 receives all required information and the inputted information meets the predetermined qualifications.

In some embodiments, the authentication system 188 compares the inputs to predetermined qualifications to determine that the healthcare providers are qualified to provide the healthcare services. The qualification standards may be based on licensure or qualification standards for entities such as insurance providers and/or government agencies. For example, in some embodiments, the qualification standards of the authentication system 188 match the standards of Medicaid. Accordingly, the authentication system 188 facilitates the healthcare providers being registered to provide healthcare services for Medicaid because the authentication system 188 performs a prescreening and collects necessary information for a Medicaid application. In some embodiments, the system 188 provides information directly to insurance providers and/or government agencies or programs.

The communication system 190 sends and receives communications with, for example, the communication systems 110 of the system 100, the communication system 172 of the user account system 136, the user device 104, and/or the healthcare provider devices 108. For example, the healthcare provider may input information via the healthcare provider devices 108 and the inputted information may be received by the communication system 190. In addition, the communication system 190 sends information relating to appointments and/or requests to the healthcare provider devices 108.

The payment system 192 receives and processes payments that are sent to the healthcare providers. For example, the users and/or insurance companies may submit payments via the payment system 126 of the healthcare management system 102 and the payment system 192 of the healthcare provider management system 124 receives a portion of the payment that is allocated for the healthcare provider. The payment system 192 may calculate the portion of the payment that is due to the healthcare provider based on insurance information, standard billing practices, healthcare services provided by the healthcare provider, and/or in any other suitable manner.

The UI generation system 194 provides a user interface to a computing device associated with the healthcare provider. For example, the user interface provided by the UI generation system 194 is displayed on the healthcare provider device 108. The user interface may include images, texts, menus, buttons, and any other suitable features. For example, the user interface may include fields for the healthcare provider to input information related to authentication of the healthcare provider and information relating to the services provided by the healthcare provider. In addition, the healthcare provider may select, via the user interface, one or more healthcare appointments to be assigned to the healthcare provider. The user interface may sort the healthcare appointments based on one or more parameters selected by the healthcare provider such as timeframe, cost, and/or healthcare services.

The appointment parameter system 198 includes parameters for assigning the appointments and, in some embodiments, assigns the appointments to the healthcare providers based on the predetermined parameters. For example, the parameters include qualification requirements, time frames, healthcare services to be provided, priority rankings, payment information, insurance information, and location information. In addition, the appointment parameter system 198 includes goals or limitations for appointments for the healthcare providers. For example, a business entity may set goals or limits for their employees using the appointment parameter system 198. Independent contractors may set their own goals or limits using the appointment parameter system 198. In some embodiments, the appointment parameter system 198 provides the parameters to the UI generation system 194 for inclusion in a user interface.

In addition, the filter system 196 of the provider management system 124 may filter the appointments 402 based on priority, length of appointment, travel time, distance, payment terms, healthcare services, specialty service requirements, or any other parameter.

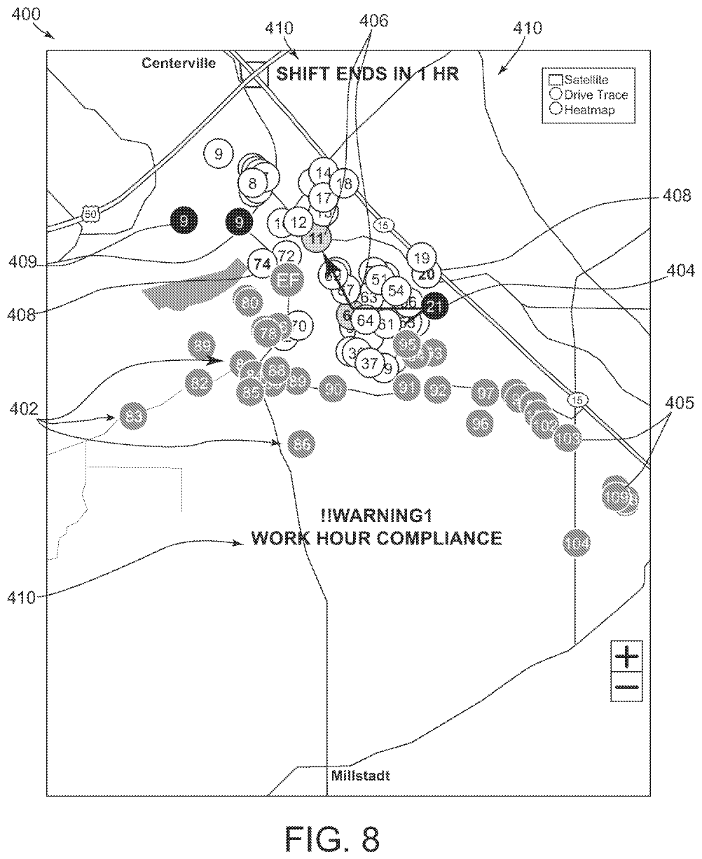

FIG. 8 illustrates an example map 400 including a plurality of healthcare appointments 402. The healthcare appointments 402 in the map 400 may be booked using the system 100. Each healthcare appointment 402 may include a patient, a healthcare service, a time frame, location information, an assignment status, payment information, contract information, and/or a priority level. In some embodiments, the status includes assigned, unassigned, or completed. The timeframe information includes an appointment window for each healthcare appointment and/or an estimated duration of the appointment. In addition, the time frame information may include a travel time for the healthcare provider to and from the appointment and/or a deadline for assigned or unassigned appointments. For example, the time frame may indicate the number of hours or minutes until an appointment is required to be completed. The payment information may include a payment amount that the healthcare provider will receive for the appointment. The contract conditions may include requirements for the appointment and/or booking information. For example, some appointments are classified as corporate appointments that are required to be assigned to a healthcare provider that is an independent contractor (i.e., a worker with a 1099 tax classification).

The healthcare appointments may be color coded on the map based on at least one of the healthcare service, time frame, the location, or the status. In further embodiments, the healthcare appointments may be sorted or filtered based on at least one of the healthcare service, time frame, the location, or the status. For example, the healthcare provider may select to view unassigned appointments and the UI generation system 194 generates a map including unassigned appointments. The system 100 can categorize or subcategorize the appointments for the user. For example, the user can select to sort or filter appointments having the same time frame by priority and/or expected appointment length.

In the example, the filter system 196 of the provider management system 124 may filter the appointments 402 that are displayed on the map based on characteristics such as priority, length of appointment, travel time, distance, payment terms, healthcare services, specialty service requirements, or any other parameter. For example, in some embodiments, the filter system 196 determines the appointments 402 that are displayed in the map 400 based on a timeframe selected by the user. For example, the filter system 196 may provide appointments 402 that are scheduled to occur within two hour time blocks. Also, the filter system 196 may filter the appointments 402 based on status, priority, or any other suitable parameter.

In the illustrated example, the appointments 402 in the map 400 are highlighted or color coded based on the status, the contract conditions, and/or the time frame. For example, the map 400 includes corporate appointments 404 that are to be assigned to independent contractors. The map 400 includes assigned appointments 405, unassigned appointments 406 that are scheduled to occur within 2 hours, and unassigned appointments 408 that are scheduled to occur in 2-6 hours. The highlighting or color coding may be selected and adjusted by a user.

In some embodiments, the user is able to draw on or edit the map 400. For example, the user may draw a route to an appointment 402. In addition, the user may highlight or select one or more of the appointments 402 in the map 400.

The appointments 402 displayed on the map 400 may be assigned by the appointment parameter system 198 based on predetermined parameters including, for example and without limitation, qualification requirements, time frames, healthcare services to be provided, priority rankings, payment information, insurance information, and location information. In addition, the appointment parameter system 198 includes goals or limitations for appointments for the healthcare providers. For example, a business entity may set goals or limits for their employees using the appointment parameter system 198. Independent contractors may set their own goals or limits using the appointment parameter system 198.

In some embodiments, the appointment parameter system 198 provides the parameters to the UI generation system 194 and the parameters are displayed on the map 400. For example, the map 400 includes messages or visual indicators 410 for the healthcare provider based on the parameters. The visual indicators 410 include work hour compliance warnings (e.g., the healthcare provider is below or above limits for hours), time frame information, geographic directions, payment information, and upcoming appointments and priority levels. The appointment parameter system 198 may provide a countdown for remaining hours in a shift or number of appointments for a healthcare provider and may request authorization from a supervisor for the healthcare provider to continue with appointments beyond a designated limit. The healthcare provider management system 124 may provide limited accessibility for the healthcare provider until the authorization is received.

The appointments 402 may be requested by the users using the user account system 136 of the system 100. For example, the user inputs an appointment request using the request system 118 and the request system 118 provides the request to the calendar system 112. The calendar system 112 provides the appointment to the UI generation system 194 of the healthcare provider management system 124 and the UI generation system 194 generates the map 400 including the appointments 402. The healthcare provider is assigned or selects the appointment using the provider management system 124. The healthcare provider receives information relating to the appointment. For example, the healthcare provider is provided with a user interface including the map 400 illustrating directions and a timeframe for the appointment 402.

The communication system 190 sends updates to patients and the healthcare service providers based on information in the map 400. For example, the location system 134 determines estimated arrival times for the healthcare provider based on the healthcare providers location and the location of the appointment and the communication system 190 provides notifications for the healthcare providers and the patients based on the estimated arrival times. For example, the communication system 190 sends notifications to the patient if the healthcare provider is off schedule or anticipated to be delayed based on a location and/or preceding appointment. In response to the notification, the patient can select to wait, reschedule, or cancel the appointment if the healthcare provider is predicted to be late for the appointment. The default setting may be for the patient to wait for the healthcare provider unless the patient responds otherwise. The communication system 190 may cause the UI generation system 194 to display an alert or provide an alarm for the healthcare service provider when the healthcare provider should leave for the next scheduled appointment and/or when the healthcare service provider is anticipated to be late for the next schedule appointment.

The location system 134 determines locations based on any location services. For example, in some embodiments, the location of the user and/or the healthcare provider is determined based on a global position system (GPS), an IP address, a cellular network signal, or any other suitable location service. The location system 134 receives the location information and correlates a location with the user and/or the healthcare provider based on the location information. The location system 134 may store and compare multiple locations. For example, the location system 134 may compare a first location of the patient to a second location of the healthcare provider and determine a distance between the locations. The location system 134 may determine a travel time between the locations and estimate an arrival time to check if the appointment will be on time.

The map 400 can indicate when appointments have been completed and are awaiting payment or have received payment. The payment system 192 may require confirmation of payment including phone verification, pin verification, signature verification and comparison, visual verification, photo identification verification, and/or pattern verification. The payment system 192 provides updates for the map 400 when payment has been received and/or confirmed.

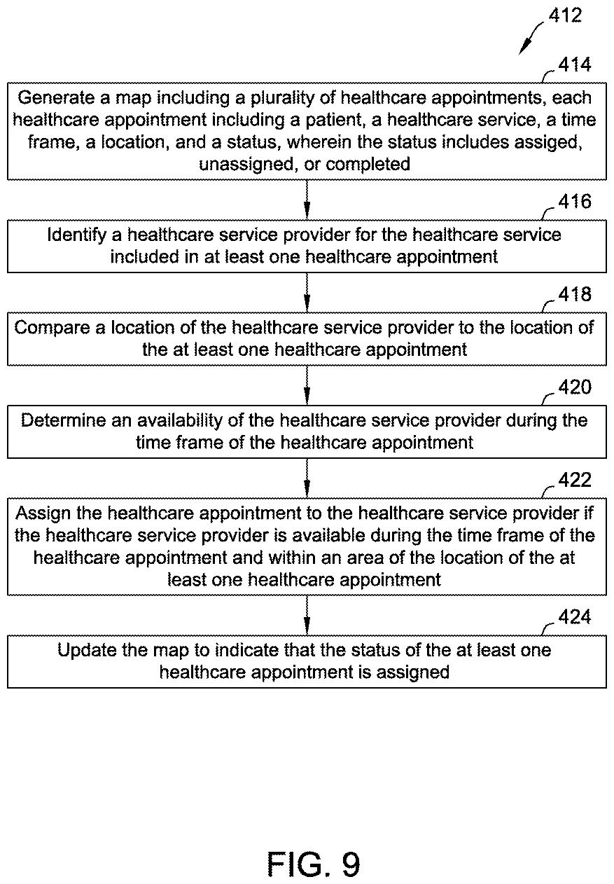

FIG. 9 is a flow diagram of an example method 412 of operating a provider management system for allocating resources for healthcare services. For example, the method 412 can be implemented using the system 100 shown in FIG. 1 and the healthcare provider management system 124 shown in FIG. 7. Referring to FIGS. 1A and 7-9, the method 412 includes generating 414 a map 400 including a plurality of healthcare appointments 402. For example, the UI generation system 194 may generate a user interface including the map 400. The map 400 may be a geographic map that represents a geographic region and includes locations. The healthcare appointments 402 may be associated with locations on the map 400. The map 400 may display any information relating to the healthcare appointments including, for example and without limitation, a patient, a healthcare service, a time frame, location information, an assignment status, payment information, contract information, and/or a priority level.

The method 412 includes identifying 416 a healthcare service provider for the healthcare service included in at least one healthcare appointment. The location system 134 compares 418 a location of the healthcare service provider to the location of the at least one healthcare appointment, and the calendar system 112 determines 420 an availability of the healthcare service provider during the time frame of the healthcare appointment.

The provider management system 124 assigns 422 the healthcare appointment to the healthcare service provider if the healthcare service provider is available during the time frame of the healthcare appointment and within an area of the location of the at least one healthcare appointment. In some embodiments, the provider management system 124 automatically assigns the healthcare appointment to the healthcare service provider meeting the criteria. In other embodiments, the healthcare provider selects or confirms the assignment using the provider management system 124. In some embodiments, the healthcare service provider is a third party contractor that agrees to provide the healthcare service based on the contract information included in the healthcare appointment.

The provider management system 124 records the assignment of the appointment and provides the assignment information to the UI generation system 194 and the calendar system 112. The UI generation system 194 updates 424 the map to indicate that the status of the at least one healthcare appointment is assigned. In addition, the calendar system 112 updates the availability of the healthcare provider based on the assigned appointment.

In some embodiments, the provider management system 124 directs the healthcare service provider to the location of the healthcare appointment within the timeframe of the healthcare appointment. For example, the location system 134 may provide a location for the appointment and the UI generation system 194 may generate a user interface with directions to the location, and estimated departure and arrival times. The provider management system 124 may account for traffic, weather, and other travel impacting factors. In some embodiments, the communication system 190 establishes a communication link between the healthcare service provider and the patient for the timeframe of the healthcare appointment.

The payment system 192 may receive at least partial payment for the healthcare appointment prior to, during, or after the timeframe of the healthcare appointment. For example, the payment system 192 may receive payment of a copay or a deposit prior to the healthcare appointment and receive a payment for the balance of the healthcare service after the appointment. In some embodiments, the payment system 192 accepts payment for the healthcare services in accord with the contract conditions and payment terms included in the healthcare appointment.

FIG. 10 is a block diagram illustrating physical components (e.g., hardware) of a computing device 500 with which aspects of the disclosure may be practiced. The computing device 500 may be integrated or otherwise associated with any of the various systems described above with respect to FIGS. 1A-1E, 7, and 10. For example, the computing device 500 may be integrated or otherwise associated with the healthcare management system 102, the user device, and/or the healthcare provider devices 108.

In a basic configuration, the computing device 500 may include at least one processing unit or processor 502 and a system memory 504. Depending on the configuration and type of computing device, the system memory 504 may comprise, but is not limited to, volatile storage (e.g., random access memory), non-volatile storage (e.g., read-only memory), flash memory, or any combination of such memories. The system memory 504 may include an operating system 514 and one or more program modules 524 or components suitable for performing the various operations described above. The operating system 514 may be suitable for controlling the operation of the computing device 500. The system memory 504 may include a healthcare management system 534 such as the healthcare management system 102 and/or one or more subsystems of the healthcare management system 102.

The computing device 500 may have additional features or functionality. For example, the computing device 500 may also include additional data storage devices (removable and/or non-removable) such as, for example, magnetic disks, optical disks, or tape. Such additional storage is illustrated in FIG. 10 by a removable storage device 544 and a non-removable storage device 554.

As stated above, a number of program modules 524 and data files may be stored in the system memory 504. While executing on the processing unit 502, the program modules 524 may perform the various processes including, but not limited to, the aspects, as described herein.

Furthermore, examples of the disclosure may be practiced in an electrical circuit comprising discrete electronic elements, packaged or integrated electronic chips containing logic gates, a circuit utilizing a microprocessor, or on a single chip containing electronic elements or microprocessors. For example, examples of the disclosure may be practiced via a system-on-a-chip (SOC) where each or many of the components illustrated in FIG. 8 may be integrated onto a single integrated circuit. Such an SOC device may include one or more processing units, graphics units, communications units, system virtualization units and various application functionality all of which are integrated (or “burned”) onto the chip substrate as a single integrated circuit.

When operating via an SOC, the functionality, described herein, with respect to the capability of client to switch protocols may be operated via application-specific logic integrated with other components of the computing device 500 on the single integrated circuit (chip). Examples of the disclosure may also be practiced using other technologies capable of performing logical operations such as, for example, “AND”, “OR”, and “NOT”, including but not limited to mechanical, optical, fluidic, and quantum technologies. In addition, examples of the disclosure may be practiced within a general-purpose computer or in any other circuits or systems.

The computing device 500 may also have one or more input/output device(s) 574. These include, but are not limited to, a keyboard, a trackpad, a mouse, a pen, a sound or voice input device, a touch, force and/or swipe input device, a display, speakers, a printer, etc. The aforementioned devices are examples and others may be used. The computing device 500 may include one or more communication systems 564 that allow or otherwise enable the computing device 500 to communicate with remote computing devices 594. Examples of suitable communication connections include, but are not limited to, radio frequency (RF) transmitter, receiver, and/or transceiver circuitry; universal serial bus (USB), parallel, and/or serial ports.

The computing device may include one or more sensors 584. The sensors may include location sensors, accelerometers, position sensors, capacitive touch sensors, the like.

The term computer-readable media as used herein may include computer storage media. Computer storage media may include volatile and nonvolatile, removable and non-removable media implemented in any method or technology for storage of information, such as computer readable instructions, data structures, or program modules.

The system memory 504, the removable storage device 544, and the non-removable storage device 554 are all computer storage media examples (e.g., memory storage). Computer storage media may include RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other article of manufacture which can be used to store information, and which can be accessed by the computing device 500. Any such computer storage media may be part of the computing device 500. Computer storage media does not include a carrier wave or other propagated or modulated data signal.

Communication media may be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media.

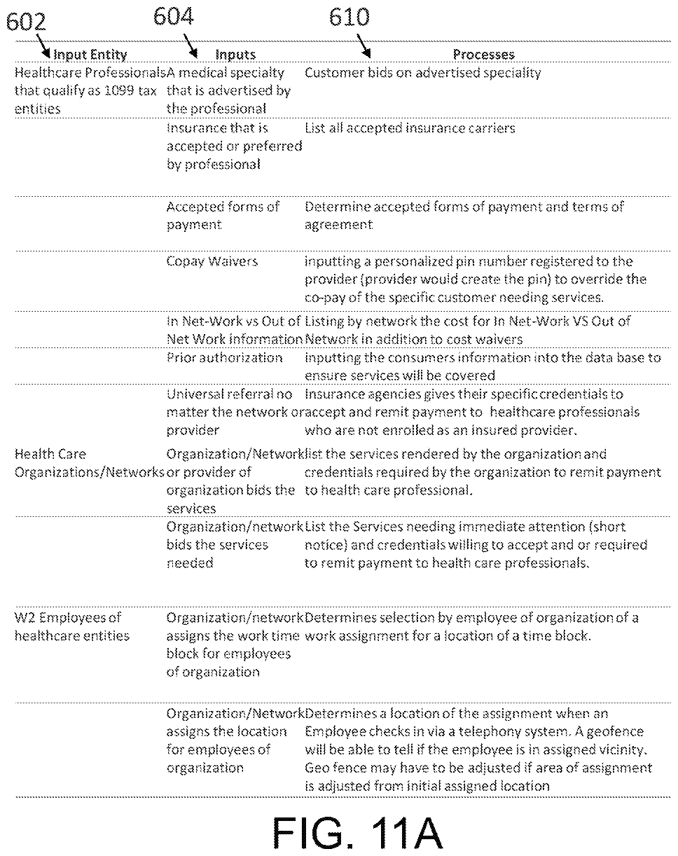

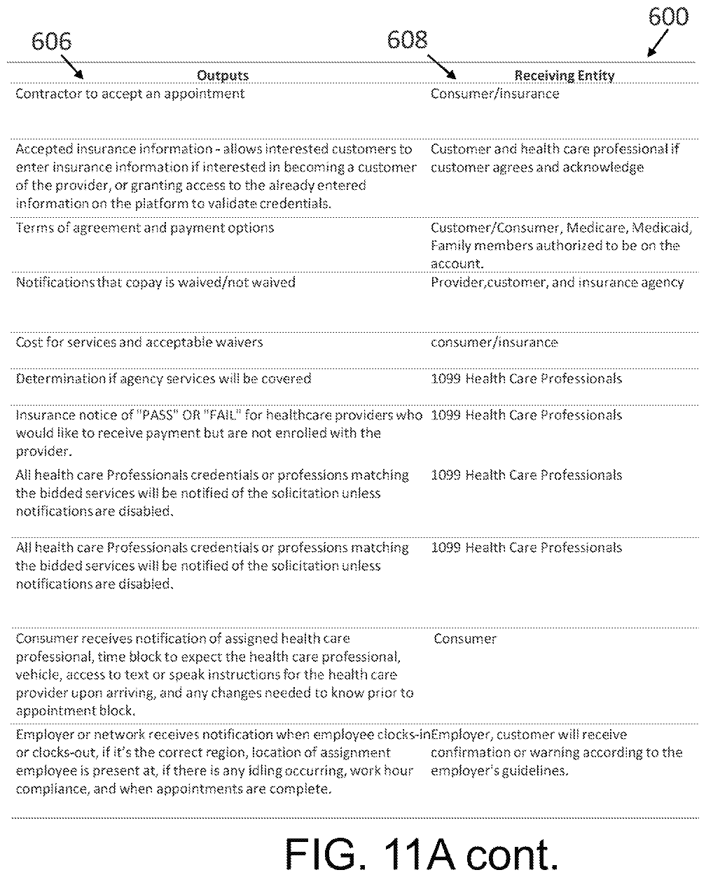

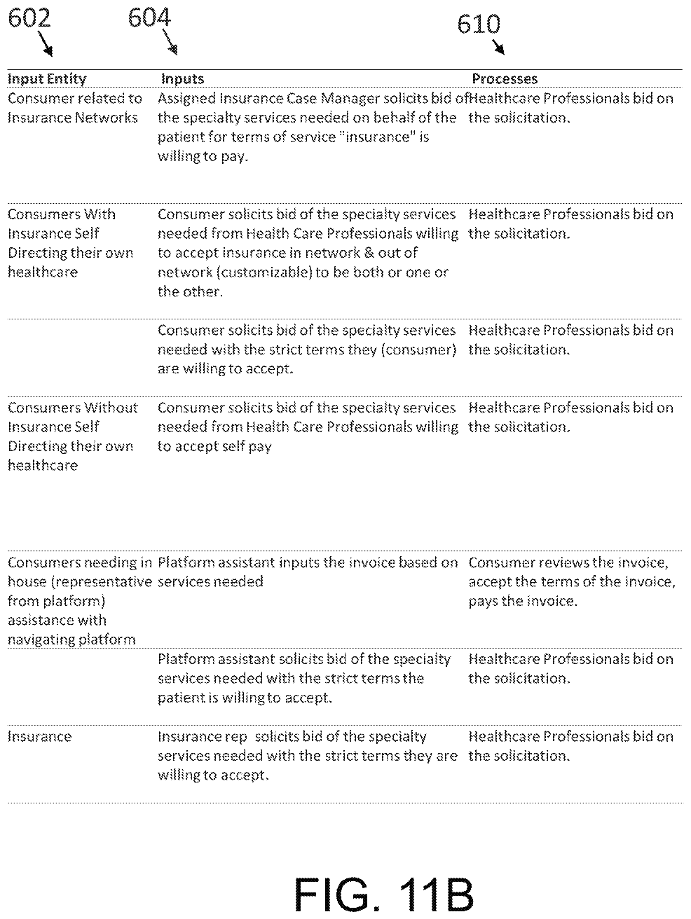

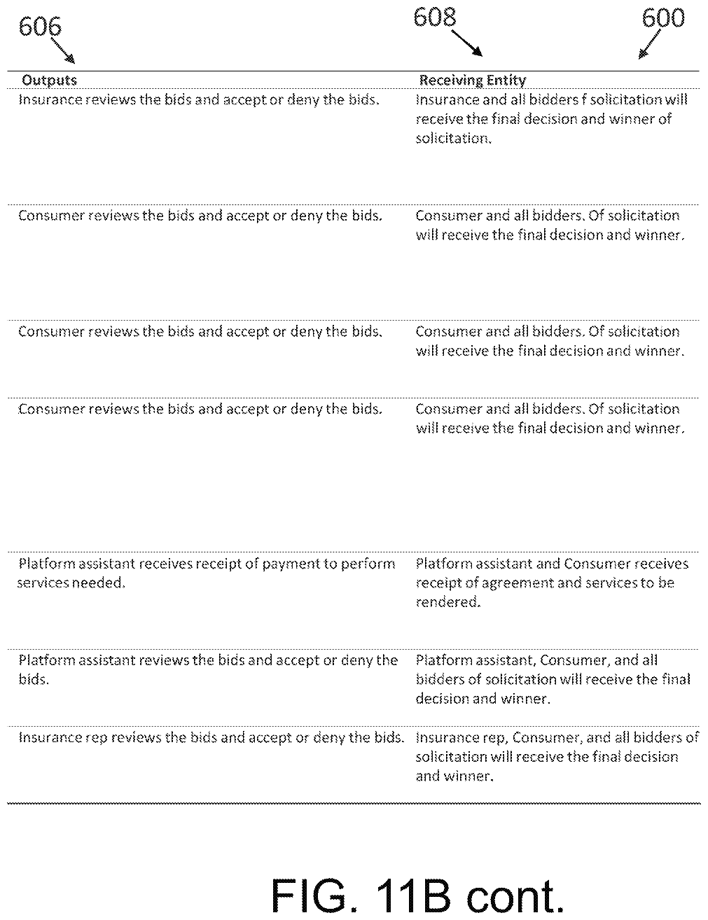

FIGS. 11A and 11B illustrate an example of a system 600 for managing healthcare services. The system 600 receives inputs 602 from computing devices associated with input entities 604 and provides outputs 606 to computing devices associated with receiving entities 608. Some parties are both input entities 604 and receiving entities 608.

Input entities 604 include, for example and without limitation, health care professionals that qualify as 1099 tax entities, healthcare organizations or networks, employees of healthcare entities, consumers related to insurance networks, consumers that have insurance but are allowed to make decisions outside of the insurance network, consumers without insurance, consumers that receive assistance navigating the system, and insurance entities.

Receiving entities 608 include, for example and without limitation, consumers or customers, insurance entities, healthcare professionals (e.g., professionals that qualify on form 1099 for federal taxes), employees, healthcare organizations, government organizations such as Medicare and Medicaid operators, authorized family members of the consumer, healthcare professionals that bid on services, platform navigation assistants, and representatives of an insurance organization.

Inputs 602 include, for example and without limitation, a medical specialty that is advertised by a professional, insurance companies or policies that are accepted or preferred by the professional, accepted forms of payment, copay waivers, in-network vs. out-of-network information, authorization information, policies regarding referrals, bids for services provided by an organization, bids for services that are required by a patient, work time or appointment information for an employee of an organization, locations of employees, solicitations of bids by an insurance company, solicitation of bids by a consumer, solicitation of bids with requirements or terms set by the consumer, solicitation of bids by a consumer who require self-pay options (e.g., consumers who are uninsured or underinsured), invoices of services needed determined with assistance of platform navigator, solicitations of bids by the platform navigator on behalf of the patient, and insurance entity solicitations of bids for healthcare services with terms required by the insurance entity.

Outputs 606 include, for example and without limitation, appointment information, accepted insurance information, terms of agreement and payment options, notifications regarding a copay, cost for services and acceptable cost waivers, determination if agency services will be covered, acceptance or rejection of unenrolled healthcare providers, solicitation notifications, scheduled appointment information, acceptance or denial of bids by insurance, acceptance or denial of bids by the consumer, receipt of payment, platform assistance acceptance or denial of bids, and acceptance or denial of bids with insurance selected terms.

The outputs 606 may be provided to the computing device related to the receiving entities 608. For example, the outputs 606 may be included in a user interface and/or an electronic message. The outputs 606 may be provided in an accessible manner to facilitate the receiving entity comprehending the outputs. For example, the outputs 606 may include readable messages, brail interpretations, and/or audio messages.

The system 600 receives the inputs 602 and performs one or more processes 610 based on the inputs 602 to generate the outputs 606. For example, consumer inputs may be received from computing devices associated with consumers who are related to insurance networks, consumers with insurance who are directing their own healthcare, consumers who do not have insurance or are underinsured, and consumers who utilize assistance with navigating the system 600. The system 600 generates solicitation requests based on the consumer inputs and determines bids from healthcare professionals based on the solicitation requests and terms from the consumer. The consumers may select or reject the bids and the system 600 notifies the healthcare professionals, the insurance providers, and the consumers of the solicitation status. The consumer selects a generated appointment when the bids are approved. For example, the system 600 provides the list of services and bid information provided by healthcare providers to a computing device associated with the customer and the customer may input a selection based on the bid information for the advertised specialty of a selected healthcare provider. The system 600 compares the bid information to information about the healthcare provider and outputs matched appointment information to the consumer and the insurance entity. The consumer and/or the insurance entity may accept or reject the matched appointments. The healthcare provider may approve or accept an appointment before the appointment is outputted to the consumer and the insurance entity.

The system 600 receives from a computing device associated with a healthcare professional the following inputs: a medical specialty that is advertised by a professional, insurance policies or companies that are accepted or preferred by the professional, accepted forms of payment, copay waivers, in-network vs. out-of-network information, authorization information, and policies regarding referrals. For example, based on inputs 602 from the healthcare organizations or networks, the system 600 generates a list of services provided by the healthcare organization and credentials required by the healthcare organization to remit payment to the healthcare professional. The system 600 compares the list of services provided and the list of requested services generated based on the consumer requests. The system 600 provides a list of requested services to a computing device associated with healthcare professionals that have credentials matching the required credentials and/or that provide the requested healthcare services. The system 600 can sort, highlight, or otherwise filter a list of requested services to indicate healthcare services requiring immediate attention. The healthcare professionals may be automatically scheduled with the matched appointments. Employees of healthcare organizations may select assignments from the list of matched appointments for their respective organization.

Based on inputs 602 from the employees of healthcare organizations, the system 600 determines a selection of a work assignment and relates the selection to a location and a time block. The system 600 provides a notification to the computing device associated with the consumer. The notification(s) may include the assigned healthcare professional, a vehicle associated with the appointment, the location, a time block to expect the healthcare professional, and any modifications or changes made to the appointment. The system 600 determines a location of the appointment when an employee checks in via a telephony system. A geofence will be able to tell if the employee is within an assigned vicinity. The geofence may be adjusted if an area of the appointment is adjusted from the initial assigned location. The system 600 provides a notification to the employer and/or the customer when the employee clocks-in or clocks-out for an appointment. The notification may include information relating to a location of the employee, a comparison of location of the employee to an assigned region, if there is any idling occurring, work hour compliance, and when appointments are complete.

Also, the system 600 generates a list of insurance providers or policies accepted by a healthcare professional and provides the insurance information to the consumer. The consumer can select the healthcare professional based on the accepted insurance information. The system 600 may provide an option for the consumer to input their insurance information or authorize transfer of the insurance information from the centralized database. In addition, the system 600 processes a copay waiver by receiving authorization information from a computing device associated with the healthcare professional such as a personalized pin number. After authorization of the copay waiver, the system 600 overrides the copay of a specified consumer requesting healthcare services. The system 600 provides the notification of the copay waiver to the consumer, the healthcare professional and the insurance provider. The insurance provider and/or the consumer may track copay waivers to determine out-of-pocket expenses and deductibles actually paid by the consumer and ensure accurate tracking towards policy limits and requirements. Some insurance providers may credit a portion of waived copays towards the policy limits. The system 600 compares in-network and out-of-network cost for services provided by healthcare professionals and applicable cost waivers and generates a list of the costs for the insurance providers and the consumers. The system 600 may process a universal waiver for using an out-of-network or unenrolled healthcare professional by receiving authorization information from a computing device associated with the insurance entity such as a personalized pin number and authorizing payment for the healthcare professional for the healthcare services. The system 600 provides a notification of approval or denial to the healthcare professionals.

The consumers may require assistance to navigate the system 600. Representatives may have expertise in navigating the system and may provide assistance to the consumers. The navigation assistants may input bid solicitations and required terms for the consumer. The navigation assistants may input or generate invoices based on the services required by the consumer.

The insurance entities may solicit bids of healthcare services based on terms that the insurance entities are willing to accept. The system 600 determines bids by the healthcare professionals for the healthcare services and generate reports for the insurance representatives of the insurance entities to review. The insurance entities deny or accept the bids and the system 600 notifies the insurance entity, the consumer, and the healthcare professionals.

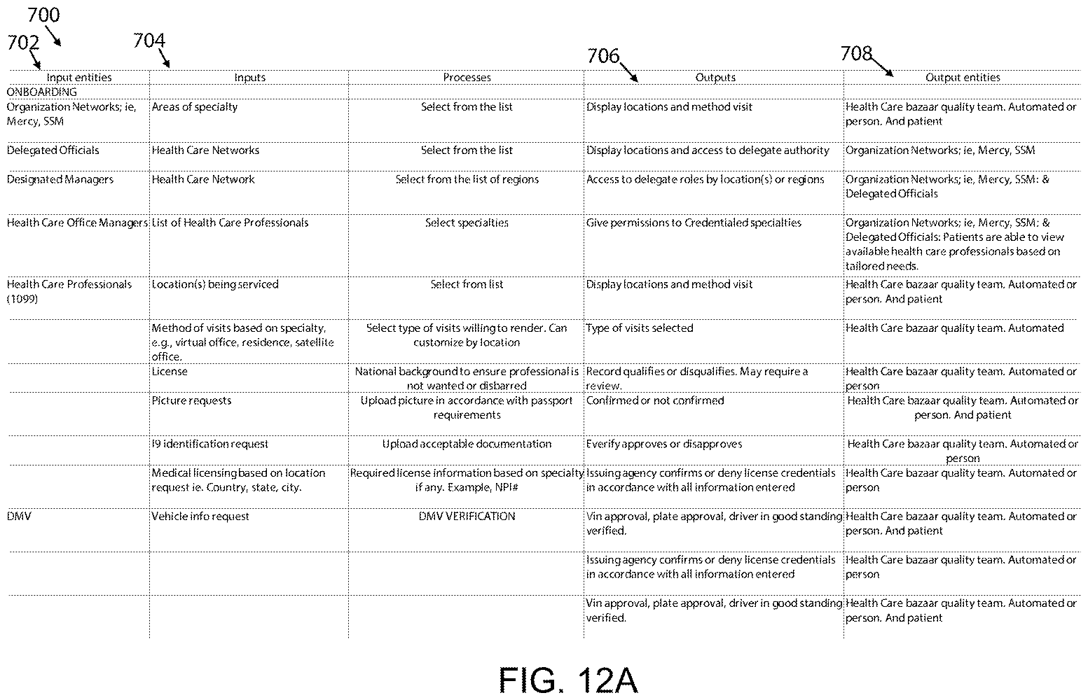

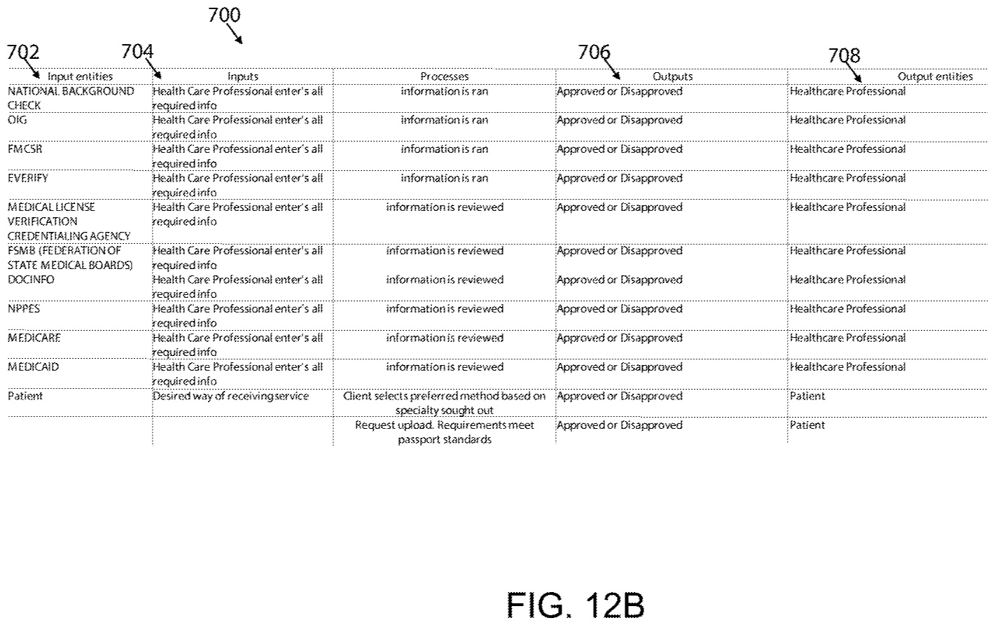

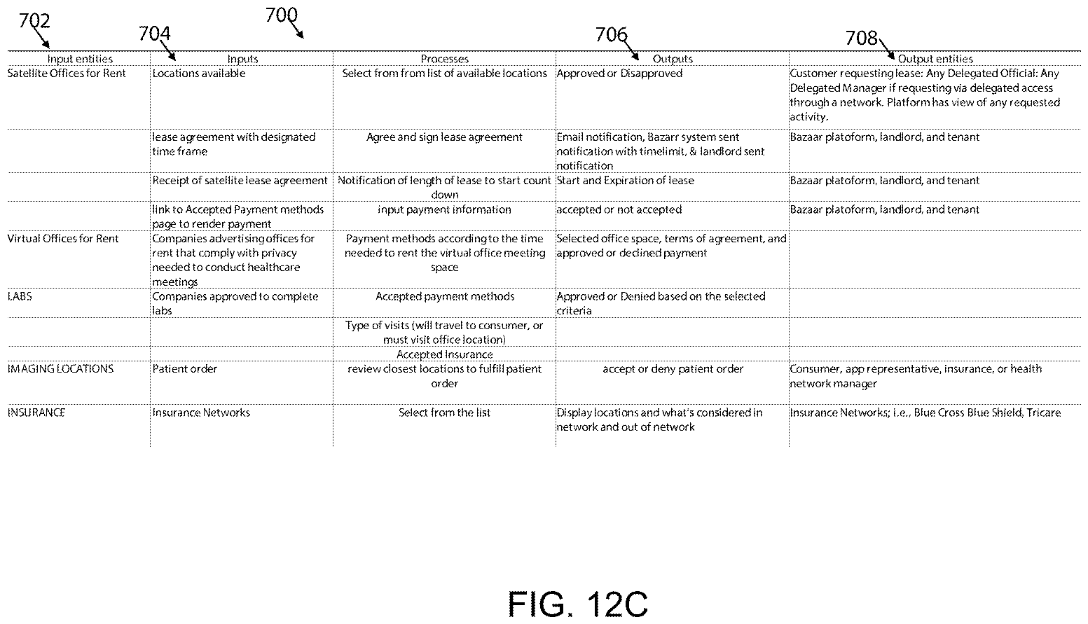

FIGS. 12A, 12B, and 12C illustrate an example of a system 700 for managing healthcare services. In particular, the system 700 provides on-boarding or enrollment services (FIG. 12A), credential verification (FIG. 12B), and property management (FIG. 12C). The system 700 may be used, for example, with the system 600 (shown in FIGS. 11A and 11B). The system 700 receives inputs 702 from computing devices associated with input entities 704 and provides outputs 706 to computing devices associated with the receiving entities 708. Some parties are both input entities 704 and receiving entities 708.

Input entities 704 include, for example and without limitation, healthcare organizations, delegated officials, designated managers, healthcare office managers, healthcare professionals, government organizations such as the department of motor vehicles (DMV), background check organizations (state or national), the office of inspector general, federal motor carrier safety regulators (FMCSR), an E-verify organization, the medical license verification credentialing agency, the federation of state medical boards, Docinfo, national plan and provider enumeration services, Medicare administrators, Medicaid administrators, patients or consumers, real estate or property parties, virtual offices, lab work providers, imaging locations, insurance providers, and an electronic data interchange (EDI).

Inputs 702 include, for example and without limitation, areas of specialty, healthcare networks, list of healthcare professionals, service locations, methods of visits based on specialty (e.g., virtual office, residence, satellite office), license information, images or photographs, identification information (e.g., information used on for form 19 identification), medical licensing based on location, vehicle information, healthcare professional information, user preferences, available locations, lease agreements with terms, receipt confirmation, payment methods, laboratory location information, proof of authorization to perform lab-work, patient orders, and insurance networks.

The system 700 processes the inputs 702 to set-up or on-board the users in the system 700, to verify credentials, and/or to determine travel or property accommodations. For example, the system receives areas of specialties and determines locations and methods of visit that are appropriate for the specialties. The system 700 receives health care networks and determines locations and access information. The system 700 receives a list of healthcare professionals, relates the specialties with the healthcare professionals, and grants permissions to credentialed specialties. The system 700 receives the location information from healthcare professionals and determines a list of locations and methods of visits that are associated with the specialties and healthcare professionals. The system 700 can determine if a healthcare professional has any issues on a background check, verify photograph requirements, document authorization to work, and store license and driving information. Also, based on the input information, the system 700 approves or disproves the healthcare professionals to provide the respective service.

The patient can select a preferred method of receiving service and the system 700 determines if the preferred method is available for the healthcare service by analyzing the healthcare professional information and the data associated with the specialty. The system 700 approves or disapprove the request.

The system 700 facilitates real estate parties (e.g., realtors, landlords, or tenants) reaching agreements on real estate transactions related to medical services. For example, the system 700 identifies properties for offices (virtual or real) or services such as imaging and laboratories based on the required terms for services, facility qualifications, published advertisements, and locations. The system 700 determines the agreement terms for the properties and sends notice to the parties of acceptance or rejection. The system 700 also facilitates payment for the services and/or property transactions.

The receiving entities 708 include, for example and without limitation, a healthcare platform representative (e.g., a quality control representative), a patient, a healthcare organization network, healthcare professionals, real estate professionals, landlords, and tenants.

Systems described herein (e.g., systems 100, 600, 700) may be used to provide education to the consumers, healthcare professionals, and/or the insurance providers. For example, the consumers may be veterans and may receive information on topics or specialties that are selected for veterans based on information provided by veteran affairs. The system may track consumers that are recognized as veterans and send them notifications of selected topics of interest. In addition, the system may send notifications to the healthcare professionals regarding selected topics of interest. The systems may provide education to consumers, insurance providers, and healthcare professionals based on information provided by any organizations such as healthcare software organizations, health services organizations, and entrepreneurs organizations. The education may be provided to consumers based on characteristics of the consumers or to all consumers using the system.

In addition, the systems (e.g., systems 100, 600, 700) may be used to provide transportation services related to healthcare services. For example, transportation (e.g., non-emergency medical transportation) may be provided to or from appointments scheduled by the systems or transportation may be provided during the course of recovery. The system receives business information of professionals enrolling a fleet into the platform for healthcare professional leasing purposes, healthcare professionals needing transportation services, and/or a list of the professional or healthcare networks. The system determines if the provider has a state-approved arbitrator agreement if verification is required, identifies a list of telephony providers, and/or determines a location(s) related to the transportation services. The system approves or disproves the provider of the transportation services. A notification of the approval/disproval is provided to the telephony company enrolling a vehicle(s) and the healthcare professional. The system then may relate the telephony service to the healthcare professional and/or the appointment and notify the patient of available transportation services for the appointment. Examples of possible transportation related services include Turo for healthcare, ride sharing for healthcare, scooters for rent (e.g. SEGWAY brand electric scooters), golf carts, all-terrain vehicles, horses, and bikes. When a type of transportation service is selected, the system determines a type of vehicle, a cost, and a time length of the transportation service.

In one example, the systems facilitate customers scheduling wellness medical retreats or other medical related services which may be covered by insurance. For example, the system receives lists of customers, lists of retreats, and insurance companies willing to pay for retreats. The system determines locations of the retreats and which retreats accept insurance. Based on stored insurance information, the system may identify insurance policies that pay a portion of the retreats. The system then calculates a cost for the customers to attend the retreats and, optionally, compares the cost to the customers budget. The system may book a retreat using the platform if the retreat is covered by an insurance policy or is within the customers budget.

Accordingly, the present disclosure describes systems and methods for managing healthcare services. Examples include a user account system that facilitates a user searching for and selecting healthcare providers based on availability and pricing information. The user is able to view prices before selecting services and may receive competitive pricing from the healthcare providers. In addition, the user account system includes primary accounts and secondary accounts that facilitate persons with secondary accounts having access to receive some healthcare services while the inputs of the secondary account are monitored, and authorization is requested from the primary account for select services. In addition, a healthcare provider management system facilitates healthcare providers efficiently allocating and assigning work. The described systems and methods provide a healthcare marketplace or bazaar that facilitates making financially informed decisions and the healthcare professionals and insurances companies bidding for healthcare services and costs. As a result, the quality and timeliness of healthcare services may be increased while decreasing costs for the healthcare services.

A method for managing a healthcare services account includes receiving login information from a user and retrieving from a memory at least one characteristic of the user based on the login information, wherein the at least one characteristic is associated with a user account designation; determining the user account designation based on the at least one characteristic of the user, wherein the at least one characteristic comprises at least one of an age, a mental capacity, a guardianship status, a grade level, a dependent status, insurance coverage, and an employment status, wherein he user account designation comprises one of a primary account and a secondary account, each secondary account is associated with at least one primary account; providing a list of healthcare services available for the user based on the user account designation; receiving a selection of at least one healthcare service from the user; requesting approval from the primary account if the associated secondary account requests a healthcare service that requires approval from the primary account for the secondary account to select the healthcare service; providing access to the selected healthcare service if the user is authorized for the healthcare service based on the user account designation or if the selection is approved by the associated primary account for the secondary account; receiving at least one input from the user related to the selected healthcare service, wherein the input from a secondary account is monitored for indications of potentially hazardous situations; and notifying the associated primary account if the inputs from the secondary account indicate a potentially hazardous situation.

A method in accordance any other paragraph further comprising accepting payment of a cost associated with the selected healthcare service for the secondary account by receiving payment from the secondary account if the selected healthcare service is included in a list of healthcare services that the secondary account can submit payment for; or receiving payment from the primary account associated with the secondary account.

A method in accordance with any other paragraph further comprising receiving confirmation from a patient that an appointment has occurred, and accepting payment from the user of a cost associated with the selected healthcare service after receiving confirmation from the patient that the appointment has occurred.

A method in accordance with any other paragraph further comprising retrieving information a medical information database that relates to the healthcare service selected by the user, and providing the information to the user.

A method in accordance with any other paragraph wherein the login information includes a login identifier and a password.

A method in accordance with any other paragraph further comprising comparing the healthcare selection to insurance information associated with the primary account or the secondary account, and requesting approval from the primary account if the secondary account selects a healthcare service from a healthcare provider that is listed out-of-network based on the insurance information.

A method for providing remote healthcare services includes receiving a search request from a patient for at least one healthcare concern, wherein the patient is associated with a first location; identifying a second location associated with the healthcare concern; retrieving a list of healthcare providers associated with the healthcare concern from a database of healthcare providers, wherein the healthcare providers are at the second location; providing to the patient the list of healthcare providers, qualification information for the healthcare providers, and data indicating a relationship between the healthcare concern and the second location; receiving a selection from the patient of a healthcare provider from the list; and establishing a communication link between the patient at the first location and the healthcare provider at the second location.

A method in accordance with any other paragraph further comprising providing identity verification information to the patient for the selected healthcare provider, wherein the identity verification information includes at least one of a photo and personal credentials.

A method in accordance with any other paragraph wherein the association between the second location and the healthcare concern comprises at least one of a number of incidents related to the healthcare concern are at the second location; research is generated at the second location and relates to the healthcare concern; and an expert in a field of the healthcare concern is at the second location.

A method for allocating resources for healthcare services includes generating a map including a plurality of healthcare appointments, each healthcare appointment including a patient, a healthcare service, a timeframe, a location, and a status, wherein the status includes assigned, unassigned, or completed; identifying a healthcare service provider that is associated with the healthcare service included in at least one healthcare appointment; comparing a location of the healthcare service provider to the location of the at least one healthcare appointment; determining an availability of the healthcare service provider during the time frame of the healthcare appointment; assigning the healthcare appointment to the healthcare service provider if the healthcare service provider is available during the time frame of the healthcare appointment and within an area of the location of the at least one healthcare appointment; and updating the map to indicate that the status of the at least one healthcare appointment is assigned.

A method in accordance with any other paragraph wherein the healthcare appointments are color coded on the map based on at least one of the healthcare service, time frame, the location, of the status.

A method in accordance with any other paragraph wherein the timeframe includes an appointment window for each healthcare appointment.

A method in accordance with any other paragraph wherein each healthcare appointment includes payment information.

A method in accordance with any other paragraph wherein each healthcare appointment includes a priority level, and further comprising sorting the healthcare appointments based on the priority level.

A method in accordance with any other paragraph wherein each healthcare appointment includes contract information including cost and payment information.

A method in accordance with any other paragraph wherein the healthcare service provider is a third party contractor that agrees to provide the healthcare service based on the contract information included in the healthcare.

A method in accordance with any other paragraph wherein the map is a geographic map, and further comprising directing the healthcare service provider to the location of the healthcare appointment within the timeframe of the healthcare appointment.

A method in accordance with any other paragraph further comprising establishing a communication link between the healthcare service provider and the patient within the timeframe of the healthcare appointment.

A method comprising receiving a search request from a patient for at least one healthcare concern; retrieving a list of healthcare providers associated with the healthcare concern from a database of healthcare providers; providing to the patient a list of healthcare services provided by the healthcare providers and a comparison of costs associated with the healthcare services provided by the healthcare providers; providing to the patient a list of available appointments for each healthcare provider; receiving a selection from the patient including a selected healthcare provider, a selected healthcare service, and a selected appointment from the list of available appointments, wherein the selected healthcare service is associated with a cost from the comparison of costs; generating an appointment booking based on the selection, wherein the appointment booking includes the selected appointment, the selected healthcare service, the selected healthcare provider, and the cost associated with the healthcare service; sending the appointment booking to the healthcare provider; receiving confirmation from the patient that the appointment has occurred; and accepting payment from the patient of the cost associated with the selected healthcare service after receiving confirmation from the patient that the appointment has occurred.

A method in accordance with any other paragraph wherein receiving confirmation from the patient that the appointment has occurred comprises receiving a patient-specific authorization code from the patient and an input from the patient that confirms that the selected healthcare services were provided.

A method in accordance with any other paragraph further comprising updating the list of available appointments based on the selected appointment.

A method in accordance with any other paragraph further comprising filtering the list of healthcare providers based on healthcare insurance data.

A method in accordance with any other paragraph further comprising providing a communication system for a virtual meeting between the patient and the selected healthcare provider at a selected appointment time.

A method in accordance with any other paragraph wherein the patient is less than 18 years old, the method further comprising receiving permission from a legal guardian of the patient for the healthcare services; receiving communications from the patient; monitoring the communications; generating suggestions for healthcare services for the patient based on the communications; and send an alert to a third party if the communications indicate a potential harm to the patient.

A method in accordance with any other paragraph further comprising receiving patient data and automatically providing a healthcare suggestion to the patient based on the patient data.

A method in accordance with any other paragraph wherein the patient data includes a patient location and wherein the healthcare suggestion is based on a healthcare issue that is associated with the patient location.

A method in accordance with any other paragraph further comprising sorting the list of healthcare providers based at least in part on the patient data.

A method in accordance with any other paragraph further comprising sending an order for a healthcare product from the healthcare provider to a healthcare product vendor after the appointment, and accepting payment from the patient for a cost of the healthcare product.

A method in accordance with any other paragraph further comprising recording an attendance or non-attendance of the patient for scheduled appointments, and restricting the patient's access to select appointments if the patient has one or more scheduled appointments that the patient did not attend.

A method in accordance with any other paragraph further comprising determining a service history and license status of each healthcare provider in the database, wherein the list of healthcare providers includes only healthcare providers with an active license status and a positive service history.

A method in accordance with any other paragraph further comprising providing the costs associated with the healthcare services to a third party and obtaining authorization from the third party for the costs before providing to the patient the list of healthcare services provided by the healthcare providers and the comparison of costs associated with the healthcare services provided by the healthcare providers.

A method in accordance with any other paragraph further comprising sending a notification to the patient that the healthcare provider has arrived at a location for the healthcare services, and receiving verification from the patient that the healthcare provider has arrived at the location.

A method in accordance with any other paragraph further comprising receiving a notification from the healthcare provider that the healthcare services have been completed, providing a verification option to the patient, and receiving verification from the patient that the healthcare services have been completed.

A method in accordance with any other paragraph further comprising sending the payment to the healthcare provider after receiving confirmation from the patient that the appointment has occurred.

A method in accordance with any other paragraph further comprising receiving a request for bids from the patient for a healthcare service, and receiving at least one bid from the healthcare providers for the healthcare service, wherein the request for bids includes insurance information and payment information, and wherein the bid from the healthcare provider includes at least an appointment availability and a cost.

A system comprising a processor; and a memory communicatively coupled to the processor and storing instructions that, when executed by the processor, perform operations, comprising: receiving a search request from a patient for at least one healthcare concern; retrieving a list of healthcare providers associated with the healthcare concern from a database of healthcare providers; providing to the patient a list of healthcare services provided by the healthcare providers and a comparison of costs associated with the healthcare services provided by the healthcare providers; providing to the patient a list of available appointments for each healthcare provider; receiving a selection from the patient including a selected healthcare provider, a selected healthcare service, and a selected appointment from the list of available appointments, wherein the selected healthcare service is associated with a cost from the comparison of costs; generating an appointment booking based on the selection, wherein the appointment booking includes the selected appointment, the selected healthcare service, the selected healthcare provider, and the cost associated with the healthcare service; sending the appointment booking to the healthcare provider; receiving confirmation from the patient that the appointment has occurred; and accepting payment from the patient of the cost associated with the selected healthcare service after receiving confirmation from the patient that the appointment has occurred.

A healthcare marketplace system comprising a processor; and a memory communicatively coupled to the processor and storing instructions that, when executed by the processor, perform operations, comprising identifying healthcare providers from a list of healthcare providers that are authorized to perform healthcare services associated with at least one healthcare concern for a patient; receiving insurance information associated with the patient; receiving bids from the healthcare providers for the healthcare services; determining out-of-pocket expenses for the patient for the healthcare services based on the insurance information and the bids by the healthcare providers; receiving an approval or rejection from the patient of at least one bid submitted by the healthcare providers; and sending an appointment booking to the patient and the respective healthcare provider if the patient approves the bid submitted by the healthcare provider.

When introducing elements of the present disclosure or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above constructions without departing from the scope of the disclosure, it is intended that all matter contained in the above description and shown in the accompanying drawings shall be interpreted as illustrative and not in a limiting sense. Although specific features of various embodiments of the disclosure may be shown in some drawings and not in others, this is for convenience only. In accordance with the principles of the disclosure, any feature of a drawing may be referenced and/or claimed in combination with any feature of any other drawing.

This written description uses examples to disclose the disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims. Regardless of whether shown and described in combination or separately, the various features (both structural and methodological) are intended to be selectively included or omitted to produce an embodiment with a particular set of features. In addition, each of the operations described above may be executed in any order. For example, one operation may be performed before another operation. Additionally, one or more of the disclosed operations may be performed simultaneously or substantially simultaneously.

What is claimed is:

1. A healthcare marketplace system comprising:
a processor; and
a memory communicatively coupled to the processor and storing instructions that, when executed by the processor, perform operations, comprising:
receiving a request for bids from a patient for a healthcare service, wherein the request for bids includes a healthcare concern for the patient and at least one of insurance information or payment information;
identifying healthcare providers from a list of healthcare providers that are authorized to perform healthcare services associated with the healthcare concern for the patient;
receiving bids from the healthcare providers for the healthcare services;
determining out-of-pocket expenses for the patient for the healthcare services based on the bids by the healthcare providers and the insurance information or the payment information;
receiving an approval or rejection from the patient of at least one bid submitted by the healthcare providers; and
sending an appointment booking to the patient and the respective healthcare provider if the patient approves the bid submitted by the healthcare provider.

2. A system in accordance with claim 1, wherein the bid from the healthcare provider includes at least an appointment availability and a cost.

3. A system in accordance with claim 1, wherein the operations comprise:
receiving confirmation from the patient that the appointment has occurred; and
accepting payment from the patient of the out-of-pocket expenses associated with the approved bid after receiving confirmation from the patient that the appointment has occurred.

4. A system in accordance with claim 1, wherein the operations comprise updating a list of available appointments for the healthcare provider based on the appointment booking.

5. A system in accordance with claim 1, wherein the operations comprise filtering the list of healthcare providers based on the insurance information.

6. A system in accordance with claim 1, further comprising a communication system configured to provide a virtual meeting between the patient and the selected healthcare provider for the appointment booking.

7. A system in accordance with claim 1, wherein the operations comprise receiving patient data and automatically providing a healthcare suggestion to the patient based on the patient data.

8. A system in accordance with claim 7, wherein the patient data includes a patient location and wherein the healthcare suggestion is based on a healthcare issue that is associated with the patient location.

9. A system in accordance with claim 7, wherein the operations comprise sorting the list of healthcare providers based at least in part on the patient data.

10. A system in accordance with claim 1, wherein the operations comprise recording an attendance or non-attendance of the patient for scheduled appointments, and restricting the patient's access to select appointments if the patient has one or more scheduled appointments that the patient did not attend.

11. A system in accordance with claim 1, wherein the operations comprise determining a service history and license status of each healthcare provider in a database, wherein the list of healthcare providers includes only healthcare providers with an active license status and a positive service history.

12. A system comprising:
a processor; and
a memory communicatively coupled to the processor and storing instructions that, when executed by the processor, perform operations, comprising:
receiving a search request from a patient for at least one healthcare concern;
retrieving a list of healthcare providers associated with the healthcare concern from a database of healthcare providers;
providing to the patient a list of healthcare services provided by the healthcare providers and a comparison of costs associated with the healthcare services provided by the healthcare providers;
providing to the patient a list of available appointments for each healthcare provider;
receiving a selection from the patient including a selected healthcare provider, a selected healthcare service, and a selected appointment from the list of available appointments, wherein the selected healthcare service is associated with a cost from the comparison of costs;
generating an appointment booking based on the selection, wherein the appointment booking includes the selected appointment, the selected healthcare service, the selected healthcare provider, and the cost associated with the healthcare service;
sending the appointment booking to the healthcare provider;
receiving confirmation from the patient that the appointment has occurred; and
accepting payment from the patient of the cost associated with the selected healthcare service after receiving confirmation from the patient that the appointment has occurred.

13. A system in accordance with claim 12, further comprising a communication system configured to provide a virtual meeting between the patient and the selected healthcare provider for the appointment booking.

14. A system in accordance with claim 12, wherein the operations comprise receiving patient data and automatically providing a healthcare suggestion to the patient based on the patient data.

15. A system in accordance with claim 12, wherein the operations comprise determining a service history and license status of each healthcare provider in a database, wherein the list of healthcare providers includes only healthcare providers with an active license status and a positive service history.

16. A method comprising:
receiving a request for bids from a patient for a healthcare service, wherein the request for bids includes a healthcare concern for the patient and at least one of insurance information or payment information;
identifying healthcare providers from a list of healthcare providers that are authorized to perform healthcare services associated with the healthcare concern for the patient;
receiving bids from the healthcare providers for the healthcare services;
determining out-of-pocket expenses for the patient for the healthcare services based on the bids by the healthcare providers and the insurance information or the payment information;
providing to the patient a list of healthcare services provided by the healthcare providers and a comparison of costs associated with the healthcare services provided by the healthcare providers;
providing to the patient a list of available appointments for each healthcare provider;
receiving a selection from the patient including a selected healthcare provider, a selected healthcare service, and a selected appointment from the list of available appointments, wherein the selected healthcare service is associated with a cost from the comparison of costs;
generating an appointment booking based on the selection, wherein the appointment booking includes the selected appointment, the selected healthcare service, the selected healthcare provider, and the cost associated with the healthcare service; and
sending the appointment booking to the healthcare provider and the patient.

17. A method in accordance with claim 16, further comprising:
receiving confirmation from the patient that the appointment has occurred; and
accepting payment from the patient of the cost associated with the selected healthcare service after receiving confirmation from the patient that the appointment has occurred.

18. A method in accordance with claim 16, further comprising receiving patient data and automatically providing a healthcare suggestion to the patient based on the patient data.

19. A method in accordance with claim 18, wherein the patient data includes a patient location and wherein the healthcare suggestion is based on a healthcare issue that is associated with the patient location.

20. A method in accordance with claim 16, further comprising determining a service history and license status of each healthcare provider in a database, wherein the list of healthcare providers includes only healthcare providers with an active license status and a positive service history.
